SEQUENCE LISTING (71 sequences)

SEQ ID NO: 1 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 213 aa, Acremonium alcalophilum
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 3 - protein, 218 aa, Aspergillus fumigatus
LPSQPEARATTVQGFDISNHQKSVNFEAAKKDGAQFVMIKATEGTTYKDTVFNSHYTGATKAGLLRGGYHFARPDKSTGSTQAKFFLKNGGGWSDDNRTLPGMLDIEYNPYGATCYGLSHSQMVAWIHDFVNEYHHATSRWPMIYTTADWWNRCTGNAKGFGDKCPLVLAAYSSSPPKTIPGDWKTWTIWQNSDKYKHGGDSDKFNGPMTQLRKLASG

SEQ ID NO: 4 - protein, 208 aa, Trichoderma reesei
TVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 5 - protein, 215 aa, Trametes cinnabarina
SPTPEKRANPKGIDVSAYQPNINWSTVKANGISFAYIKATEGTTYTNPDFSSQYTGATNAGLIRGGYHFAHPDSSSGATQAKYFLAHGGGWTSDGITLPGALDIEYNPSGAECYGLSASAMVSWIKDFSNTYHSSTGVYPVIYTTTDWWTTCTGNSAAFASTNPLWIARYASSIGTLPAGWSYTTFWQYADSGPNPGDQDEFNGSMAGLKQLALG

SEQ ID NO: 6 - protein, 207 aa, Sporormia fimetaria
AVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 7 - protein, 201 aa, Poronia punctata
AVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 8 - protein, 201 aa, Poronia punctata
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 9 - protein, 203 aa, Lecanicillium sp.
VDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 10 - protein, 208 aa, Lecanicillium sp.
TVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 11 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 12 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 13 - protein, 207 aa, Trichobolus zukalii
AVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 14 - protein, 207 aa, Penicillium citrinum
LIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 15 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 16 - protein, 208 aa, Umbelopsis westeae
KLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 17 - protein, 208 aa, Zygomycetes sp.
TLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 18 - protein, 206 aa, Chaetomium cupreum
TVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 19 - protein, 207 aa, Cordyceps cardinalis
LIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 20 - protein, 216 aa, Penicillium sp.
STIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 21 - protein, 218 aa, Aspergillus sp.
LPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 22 - protein, 204 aa, Paecilomyces sp.
TVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 23 - protein, 203 aa, Paecilomyces sp.
AVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 24 - protein, 208 aa, Pycnidiophora sp.
AVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 25 - protein, 210 aa, Thermomucor sp.
YQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 26 - protein, 207 aa, Isaria farinosa
LTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 27 - protein, 207 aa, Lecanicillium sp.
SVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 28 - protein, 208 aa, Zopfiella sp.
AVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 29 - protein, 217 aa, Malbranchea flava
GPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 30 - protein, 208 aa, Hypholoma polytrichi
LVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 31 - protein, 201 aa, Aspergillus deflectus
AVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 32 - protein, 202 aa, Ascobolus stictoideus
AVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 33 - protein, 207 aa, Coniochaeta sp.
TVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 34 - protein, 202 aa, Daldinia fissa
AVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 35 - protein, 201 aa, Rosellinia sp.
AVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 36 - protein, 202 aa, Ascobolus sp.
AVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 37 - protein, 206 aa, Curreya sp.
TVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 38 - protein, 202 aa, Coniothyrium sp.
AVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 39 - protein, 202 aa, Hypoxylon sp.
AVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 40 - protein, 202 aa, Xylariaceae sp.
AVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 41 - protein, 202 aa, Hypoxylon sp.
AVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 42 - protein, 206 aa, Yunnania penicillata
DVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 43 - protein, 207 aa, Engyodontium album
RVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 44 - protein, 208 aa, Metapochonia bulbillosa
TVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 45 - protein, 215 aa, Hamigera paravellanea
APLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 46 - protein, 217 aa, Metarhizium iadini
SPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 47 - protein, 214 aa, Thermoascus aurantiacus
ELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 48 - protein, 208 aa, Clonostachys rossmaniae
AVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 49 - protein, 203 aa, Simplicillium obclavatum
APKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 50 - protein, 216 aa, Aspergillus inflatus
APLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 51 - protein, 207 aa, Paracremonium inflatum
KVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 52 - protein, 208 aa, Westerdykella sp.
AVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 53 - protein, 207 aa, Stropharia semiglobata
LTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 54 - protein, 208 aa, Gelasinospora cratophora
TVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 55 - protein, 207 aa, Flammulina velutipes
RLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 56 - protein, 207 aa, Deconica coprophila
LVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 57 - protein, 208 aa, Rhizomucor pusillus
YETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 58 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 59 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 60 - protein, 207 aa, Myceliophthora fergusii
AVQGFDISHWQSSVDFKAAYNSGARFVIIKATEGTSFIDPKFSSHYTGATNAGFIRGAYHFAHPGQSSGEAQADYFLAHGGGWTPDGITLPGMLDLEAYNAGECWGLSQSAMVAWIKAFSDRYHARTGVYPMLYTNLSWWKTCTGNSKAFVNTNPLVLARWASSPGEIPGGWPWQTIWQNSDSYRYGGDSDIFNGDMNQLRRLATAA

SEQ ID NO: 61 - protein, 204 aa, Mortierella alpina
ALPKGIDVSHWQGDVNWNSVKAAGIEFVYIKATESINYIDSKFDANYVGATNAGLIRGGYHFARPAASSGAVQANYFLANGGGWSSDGITLPGALDLEAGCSGLSQAAMTAWVRDFSDTYHARTGRYPVIYTTTSWWKQCTGNASGFQNNNPLWIARWASSAGELPAGYAFHTFWQYADKGPNPGDQDYFNGDSAGLRRFAKGS

SEQ ID NO: 62 - protein, 216 aa, Penicillium atrovenetum
TPLESRASGVQGFDISSYQGTVDFAGAYAAGARFVMIKATEGTTYTDKTFSSHYEGASSAGLIRGGYHFAHPDSSSGAKQAEYFLAHGGGWSNDGKTLPGMLDIEYNPSGATCYGISKSAMVAWVKDFGETYKGKTGRYPMIYTTADWWNTCTGGSTAFSKDYPLVLARYSSSVGTIPGGWPYQSFWQNSDKYTYGGDSDLWNGSEASLKTFAKGA

SEQ ID NO: 63 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 64 - protein, 249 aa, Chaetomium thermophilum
PASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 65 - protein, 248 aa, Trichoderma harzianum
YPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 66 - protein, 245 aa, Trichophaea minuta
YPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 67 - protein, 249 aa, Chaetomium sp.
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 68 - protein, 245 aa, Mortierella sp.
YPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 69 - protein, 247 aa, Metarhizium sp.
YPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 70 - protein, 250 aa, Geomyces auratus
AFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 71 - protein, 240 aa, Ilyonectria rufa
YKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC

(19) United States
(12) Patent Application Publication
(10) Pub. No.: US 2026/0231983 A1
(43) Pub. Date: Aug. 13, 2026

(54) ANIMAL FEED COMPOSITIONS AND USES THEREOF

(71) Applicant: Novonesis Animal Biosolutions AG, Sisseln AG (CH)

(72) Inventors: Leticia CARDOSO BITTENCOURT, Kaiseraugst (CH); Rual LOPEZ-ULIBARRI, Kaiseraugst (CH); Estefania PEREZ CALVO, Kaiseraugst (CH)

(73) Assignee: NOVOZYMES A/S, Bagsvaerd (DK)

(21) Appl. No.: 19/304,093

(22) Filed: Aug. 19, 2025

Related U.S. Application Data
(62) Division of application No. 17/275,903, filed on Sep. 17, 2019, now Pat. No. 12,439,939.

(30) Foreign Application Priority Data
Sep. 17, 2018 (EP) 18194834.0

(51) Int. Cl. A23K 20/189; A23K 10/22; A23K 10/26; A23K 20/142; A23K 20/174; A23K 50/30; A23K 50/40; A23K 50/75; A23K 50/80

(52) U.S. Cl. CPC A23K 20/189; A23K 10/22; A23K 10/26; A23K 20/142; A23K 20/174; A23K 50/30; A23K 50/40; A23K 50/75; A23K 50/80; C12Y 302/01017

(57) ABSTRACT

The present invention relates to a composition and/or an animal feed comprising polypeptides having muramidase activity and essential oils and uses thereof.

### CROSS-REFERENCE TO RELATED APPLICATIONS

This application is a divisional of U.S. application Ser. No. 17/275,903 filed Mar. 12, 2021 (now U.S. Pat. No. 12,439,939), which in turn is the U.S. national phase of International Application No. PCT/EP202019,74775 filed Sep. 17, 2019 which designated the U.S. and claims priority to EP patent application Ser. No. 18/194,834.0 filed Sep. 17, 2018, the entire contents of each of which are hereby incorporated by reference.

### REFERENCE TO A SEQUENCE LISTING

This application contains a Sequence Listing (Name: 14981-US-PCD Sequence Listing.xml; Size: 76,987 bytes; and Date of Creation: Apr. 21, 2026) which is incorporated herein by reference in its entirety.

### BACKGROUND OF THE INVENTION

### Field of the Invention

The present invention relates to a composition and an animal feed comprising polypeptides having muramidase activity and essential oils and uses thereof.

### Description of the Related Art

Muramidase, also named as lysozyme, is an O-glycosyl hydrolase produced as a defensive mechanism against bacteria by many organisms. The enzyme causes the hydrolysis of bacterial cell walls by cleaving the glycosidic bonds of peptidoglycan; an important structural molecule in bacteria. After having their cell walls weakened by muramidase action, bacterial cells lyse as a result of umbalanced osmotic pressure.

Muramidase naturally occurs in many organisms such as viruses, plants, insects, birds, reptiles and mammals. In mammals, Muramidase has been isolated from nasal secretions, saliva, tears, intestinal content, urine and milk. The enzyme cleaves the glycosidic bond between carbon number 1 of N-acetylmuramic acid and carbon number 4 of N-acetyl-D-glucosamine. In vivo, these two carbohydrates are polymerized to form the cell wall polysaccharide of many microorganisms.

Muramidase has been classified into five different glycoside hydrolase (GH) families (CAZy, www.cazy.org): hen egg-white muramidase (GH22), goose egg-white muramidase (GH23), bacteriophage T4 muramidase (GH24), Sphingomonas flagellar protein (GH73) and Chalaropsis muramidases (GH25). Muramidase extracted from hen egg white (a GH22 muramidase) is the primary product available on the commercial market, and traditionally has just been referred to as muramidase even though nowadays there are many other known muramidases.

Improving the growth performance of farm animals is needed in a world with a growing population eating more animal protein, and it is the object of the present invention to devise solutions which helps meet this challenge.

### SUMMARY OF THE INVENTION

The present invention relates to a composition comprising one or more polypeptides having muramidase activity and one or more essential oils.

The present invention also relates to an animal feed comprising one or more polypeptides having muramidase activity and one or more essential oils.

The present invention further relates to a method of improving body weight gain (BWG), feed conversion ratio (FCR), carotenoid absorption and/or intestinal permeability in an animal comprising administering to an animal the composition or the animal feed of the present invention.

The present invention further relates to use of the composition or the animal feed of the present invention in improving BWG, FCR, carotenoid absorption and/or intestinal permeability in an animal.

### Overview of Sequence Listing

SEQ ID NO: 1 is the mature amino acid sequence of a GH25 muramidase from Acremonium alcalophilum as described in WO2013/076253 (SEQ ID NO: 4).

SEQ ID NO: 2 is the mature amino acid sequence of a GH25 muramidase from Acremonium alcalophilum as described in WO2013/076253 (SEQ ID NO: 8).

SEQ ID NO: 3 is the mature amino acid sequence of a GH25 muramidase from Aspergillus fumigatus as described in WO2011/104339 (SEQ ID NO: 3).

SEQ ID NO: 4 is the mature amino acid sequence of a GH25 muramidase from Trichoderma reesei as described in WO2009/102755 (SEQ ID NO: 4).

SEQ ID NO: 5 is the mature amino acid sequence of a GH25 muramidase from Trametes cinnabarina as described in WO2005/080559 (SEQ ID NO: 2).

SEQ ID NO: 6 is the mature amino acid sequence of a GH25 muramidase from Sporormia fimetaria as described in PCT/CN2017/075978 (SEQ ID NO: 3).

SEQ ID NO: 7 is the mature amino acid sequence of a GH25 muramidase from Poronia punctata as described in PCT/CN2017/075978 (SEQ ID NO: 6).

SEQ ID NO: 8 is the mature amino acid sequence of a GH25 muramidase from Poronia punctata as described in PCT/CN2017/075978 (SEQ ID NO: 9).

SEQ ID NO: 9 is the mature amino acid sequence of a GH25 muramidase from Lecanicillium sp. WMM742 as described in PCT/CN2017/075978 (SEQ ID NO: 12).

SEQ ID NO: 10 is the mature amino acid sequence of a GH25 muramidase from Lecanicillium sp. WMM742 as described in PCT/CN2017/075978 (SEQ ID NO: 15).

SEQ ID NO: 11 is the mature amino acid sequence of a GH25 muramidase from Onygena equina as described in PCT/CN2017/075978 (SEQ ID NO: 18).

SEQ ID NO: 12 is the mature amino acid sequence of a GH25 muramidase from Purpureocillium lilacinum as described in PCT/CN2017/075978 (SEQ ID NO: 21).

SEQ ID NO: 13 is the mature amino acid sequence of a GH25 muramidase from Trichobolus zukalii as described in PCT/CN2017/075978 (SEQ ID NO: 24).

SEQ ID NO: 14 is the mature amino acid sequence of a GH25 muramidase from Penicillium citrinum as described in PCT/CN2017/075978 (SEQ ID NO: 27).

SEQ ID NO: 15 is the mature amino acid sequence of a GH25 muramidase from Cladorrhinum bulbillosum as described in PCT/CN2017/075978 (SEQ ID NO: 30).

SEQ ID NO: 16 is the mature amino acid sequence of a GH25 muramidase from Umbelopsis westeae as described in PCT/CN2017/075978 (SEQ ID NO: 33).

SEQ ID NO: 17 is the mature amino acid sequence of a GH25 muramidase from Zygomycetes sp. XZ2655 as described in PCT/CN2017/075978 (SEQ ID NO: 36).

SEQ ID NO: 18 is the mature amino acid sequence of a GH25 muramidase from Chaetomium cupreum as described in PCT/CN2017/075978 (SEQ ID NO: 39).

SEQ ID NO: 19 is the mature amino acid sequence of a GH25 muramidase from Cordyceps cardinalis as described in PCT/CN2017/075978 (SEQ ID NO: 42).

SEQ ID NO: 20 is the mature amino acid sequence of a GH25 muramidase from Penicillium sp. ‘qil’ as described in PCT/CN2017/075978 (SEQ ID NO: 45).

SEQ ID NO: 21 is the mature amino acid sequence of a GH25 muramidase from Aspergillus sp. nov XZ2609 as described in PCT/CN2017/075978 (SEQ ID NO: 48).

SEQ ID NO: 22 is the mature amino acid sequence of a GH25 muramidase from Paecilomyces sp. XZ2658 as described in PCT/CN2017/075978 (SEQ ID NO: 51).

SEQ ID NO: 23 is the mature amino acid sequence of a GH25 muramidase from Paecilomyces sp. XZ2658 as described in PCT/CN2017/075978 (SEQ ID NO: 54).

SEQ ID NO: 24 is the mature amino acid sequence of a GH25 muramidase from Pycnidiophora cf dispera as described in PCT/CN2017/075978 (SEQ ID NO: 60).

SEQ ID NO: 25 is the mature amino acid sequence of a GH25 muramidase from Thermomucor indicae-seudaticae as described in PCT/CN2017/075978 (SEQ ID NO: 63).

SEQ ID NO: 26 is the mature amino acid sequence of a GH25 muramidase from Isaria farinosa as described in PCT/CN2017/075978 (SEQ ID NO: 66).

SEQ ID NO: 27 is the mature amino acid sequence of a GH25 muramidase from Lecanicillium sp. WMM742 as described in PCT/CN2017/075978 (SEQ ID NO: 69).

SEQ ID NO: 28 is the mature amino acid sequence of a GH25 muramidase from Zopfiella sp. t180-6 as described in PCT/CN2017/075978 (SEQ ID NO: 72).

SEQ ID NO: 29 is the mature amino acid sequence of a GH25 muramidase from Malbranchea flava as described in PCT/CN2017/075978 (SEQ ID NO: 75).

SEQ ID NO: 30 is the mature amino acid sequence of a GH25 muramidase from Hypholoma polytrichi as described in PCT/CN2017/075978 (SEQ ID NO: 80).

SEQ ID NO: 31 is the mature amino acid sequence of a GH25 muramidase from Aspergillus deflectus as described in PCT/CN2017/075978 (SEQ ID NO: 83).

SEQ ID NO: 32 is the mature amino acid sequence of a GH25 muramidase from Ascobolus stictoideus as described in PCT/CN2017/075978 (SEQ ID NO: 86).

SEQ ID NO: 33 is the mature amino acid sequence of a GH25 muramidase from Coniochaeta sp. as described in PCT/CN2017/075978 (SEQ ID NO: 89).

SEQ ID NO: 34 is the mature amino acid sequence of a GH25 muramidase from Daldinia fissa as described in PCT/CN2017/075978 (SEQ ID NO: 92).

SEQ ID NO: 35 is the mature amino acid sequence of a GH25 muramidase from Rosellinia sp. as described in PCT/CN2017/075978 (SEQ ID NO: 95).

SEQ ID NO: 36 is the mature amino acid sequence of a GH25 muramidase from Ascobolus sp. ZY179 as described in PCT/CN2017/075978 (SEQ ID NO: 98).

SEQ ID NO: 37 is the mature amino acid sequence of a GH25 muramidase from Curreya sp. XZ2623 as described in PCT/CN2017/075978 (SEQ ID NO: 101).

SEQ ID NO: 38 is the mature amino acid sequence of a GH25 muramidase from Coniothyrium sp. as described in PCT/CN2017/075978 (SEQ ID NO: 104).

SEQ ID NO: 39 is the mature amino acid sequence of a GH25 muramidase from Hypoxylon sp. as described in PCT/CN2017/075978 (SEQ ID NO: 107).

SEQ ID NO: 40 is the mature amino acid sequence of a GH25 muramidase from Xylariaceae sp. 1653 h as described in PCT/CN2017/075978 (SEQ ID NO: 110).

SEQ ID NO: 41 is the mature amino acid sequence of a GH25 muramidase from Hypoxylon sp. as described in PCT/CN2017/075978 (SEQ ID NO: 113).

SEQ ID NO: 42 is the mature amino acid sequence of a GH25 muramidase from Yunnania penicillata as described in PCT/CN2017/075978 (SEQ ID NO: 116).

SEQ ID NO: 43 is the mature amino acid sequence of a GH25 muramidase from Engyodontium album as described in PCT/CN2017/075978 (SEQ ID NO: 119).

SEQ ID NO: 44 is the mature amino acid sequence of a GH25 muramidase from Metapochonia bulbillosa as described in PCT/CN2017/075978 (SEQ ID NO: 122).

SEQ ID NO: 45 is the mature amino acid sequence of a GH25 muramidase from Hamigera paravellanea as described in PCT/CN2017/075978 (SEQ ID NO: 125).

SEQ ID NO: 46 is the mature amino acid sequence of a GH25 muramidase from Metarhizium iadini as described in PCT/CN2017/075978 (SEQ ID NO: 128).

SEQ ID NO: 47 is the mature amino acid sequence of a GH25 muramidase from Thermoascus aurantiacus as described in PCT/CN2017/075978 (SEQ ID NO: 131).

SEQ ID NO: 48 is the mature amino acid sequence of a GH25 muramidase from Clonostachys rossmaniae as described in PCT/CN2017/075978 (SEQ ID NO: 134).

SEQ ID NO: 49 is the mature amino acid sequence of a GH25 muramidase from Simplicillium obclavatum as described in PCT/CN2017/075978 (SEQ ID NO: 137).

SEQ ID NO: 50 is the mature amino acid sequence of a GH25 muramidase from Aspergillus inflatus as described in PCT/CN2017/075978 (SEQ ID NO: 140).

SEQ ID NO: 51 is the mature amino acid sequence of a GH25 muramidase from Paracremonium inflatum as described in PCT/CN2017/075978 (SEQ ID NO: 143).

SEQ ID NO: 52 is the mature amino acid sequence of a GH25 muramidase from Westerdykella sp. as described in PCT/CN2017/075978 (SEQ ID NO: 146).

SEQ ID NO: 53 is the mature amino acid sequence of a GH25 muramidase from Stropharia semiglobata as described in PCT/CN2017/075978 (SEQ ID NO: 155).

SEQ ID NO: 54 is the mature amino acid sequence of a GH25 muramidase from Gelasinospora cratophora as described in PCT/CN2017/075978 (SEQ ID NO: 158).

SEQ ID NO: 55 is the mature amino acid sequence of a GH25 muramidase from Flammulina velutipes as described in PCT/CN2017/075978 (SEQ ID NO: 221).

SEQ ID NO: 56 is the mature amino acid sequence of a GH25 muramidase from Deconica coprophila as described in PCT/CN2017/075978 (SEQ ID NO: 224).

SEQ ID NO: 57 is the mature amino acid sequence of a GH25 muramidase from Rhizomucor pusillus as described in PCT/CN2017/075978 (SEQ ID NO: 227).

SEQ ID NO: 58 is the mature amino acid sequence of a GH25 muramidase from Stropharia semiglobata as described in PCT/CN2017/075978 (SEQ ID NO: 230).

SEQ ID NO: 59 is the mature amino acid sequence of a GH25 muramidase from Stropharia semiglobata as described in PCT/CN2017/075978 (SEQ ID NO: 233).

SEQ ID NO: 60 is the mature amino acid sequence of a GH25 muramidase from Myceliophthora fergusii as described in PCT/CN2017/075960 (SEQ ID NO: 3).

SEQ ID NO: 61 is the mature amino acid sequence of a GH25 muramidase from Mortierella alpina as described in PCT/CN2017/075960 (SEQ ID NO: 15).

SEQ ID NO: 62 is the mature amino acid sequence of a GH25 muramidase from Penicillium atrovenetum as described in PCT/CN2017/075960 (SEQ ID NO: 27).

SEQ ID NO: 63 is the mature amino acid sequence of a GH24 muramidase from Trichophaea saccata as described in WO2017/000922 (SEQ ID NO: 257).

SEQ ID NO: 64 is the mature amino acid sequence of a GH24 muramidase from Chaetomium thermophilum as described in WO2017/000922 (SEQ ID NO: 264).

SEQ ID NO: 65 is the mature amino acid sequence of a GH24 muramidase from Trichoderma harzianum as described in WO2017/000922 (SEQ ID NO: 267).

SEQ ID NO: 66 is the mature amino acid sequence of a GH24 muramidase from Trichophaea minuta as described in WO2017/000922 (SEQ ID NO: 291).

SEQ ID NO: 67 is the mature amino acid sequence of a GH24 muramidase from Chaetomium sp. ZY287 as described in WO2017/000922 (SEQ ID NO: 294).

SEQ ID NO: 68 is the mature amino acid sequence of a GH24 muramidase from Mortierella sp. ZY002 as described in WO2017/000922 (SEQ ID NO: 297).

SEQ ID NO: 69 is the mature amino acid sequence of a GH24 muramidase from Metarhizium sp. XZ2431 as described in WO2017/000922 (SEQ ID NO: 300).

SEQ ID NO: 70 is the mature amino acid sequence of a GH24 muramidase from Geomyces auratus as described in WO2017/000922 (SEQ ID NO: 303).

SEQ ID NO: 71 is the mature amino acid sequence of a GH24 muramidase from Ilyonectria rufa as described in WO2017/000922 (SEQ ID NO: 306).

### FIGURES

FIG. 1: Intestinal integrity assessed by passage of Dextran-FITC. Each point represents an animal and the crossbars represent the mean of each group. Different letters differ by Tukey test (P<0.05).

### DEFINITIONS

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pets such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Body Weight Gain: The term “body weight gain” means an increase in live weight of an animal during a given period of time e.g. the increase in weight from day 1 to day 21.

Feed Conversion Ratio (FCR): FCR is a measure of an animal's efficiency in converting feed mass into increases of the desired output. Animals raised for meat—such as swine, poultry and fish—the output is the mass gained by the animal. Specifically FCR is calculated as feed intake divided by weight gain, all over a specified period. Improvement in FCR means reduction of the FCR value. A FCR improvement of 2% means that the FCR was reduced by 2%.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide or a catalytic domain having one or more (e.g., several) amino acids absent from the amino and/or carboxyl terminus of a mature polypeptide or domain; wherein the fragment has muramidase activity.

In one aspect, a fragment of a GH24 muramidase (such as one of SEQ ID NO: 63 to 71) comprises at least 230 amino acids, such as at least 235 amino acids, at least 240 amino acids, or at least 245 amino acids and has muramidase activity. In another aspect, a fragment of a GH24 muramidase (such as one of SEQ ID NO: 63 to 71) comprises at least 90% of the length of the mature polypeptide, such as at least 92%, at least 94%, at least 96%, at least 98% or at least 99% of the length of the mature polypeptide and has muramidase activity.

In one aspect, a fragment of a GH25 muramidase (such as one of SEQ ID NO: 1 to 72) comprises at least 180 amino acids, such as at least 185 amino acids, at least 190 amino acids, at least 195 amino acids, at least 200 amino acids, at least 205 amino acids or at least 210 amino acids and has muramidase activity. In another aspect, a fragment of a GH25 muramidase (such as one of SEQ ID NO: 1 to 72) comprises at least 90% of the length of the mature polypeptide, such as at least 92%, at least 94%, at least 96%, at least 98% or at least 99% of the length of the mature polypeptide and has muramidase activity.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Muramidase activity: The term “muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of muramidase. In appropriate experimental conditions these changes are proportional to the amount of muramidase in the medium (c.f. INS 1105 of the Combined Compendium of Food Additive Specifications of the Food and Agriculture Organisation of the UN (www.fao.org)). For the purpose of the present invention, muramidase activity is determined according to the turbidity assay described in example 1 (“Determination of Muramidase Activity”) and the polypeptide has muramidase activity if it shows activity against one or more bacteria, such as Micrococcus luteus ATCC 4698 and/or Exiguobacterium undea (DSM14481). As an example, the GH25 muramidase of the present invention has at least 20%, e.g., at least 40%, at least 50%, at least 60%, at least 70%, at least 80%, at least 90%, at least 95%, or at least 100% of the muramidase activity of SEQ ID NO: 1. As another example, the GH24 muramidase of the present invention have at least 20%, e.g., at least 40%, at least 50%, at least 60%, at least 70%, at least 80%, at least 90%, at least 95%, or at least 100% of the muramidase activity of SEQ ID NO: 63.

Mature polypeptide: The term “mature polypeptide” means a polypeptide in its final form following translation and any post-translational modifications, such as N-terminal processing, C-terminal truncation, glycosylation, phosphorylation, etc.

In the present invention, the mature polypeptide may be amino acids 1 to 208 of SEQ ID NO: 1, amino acids 1 to 213 of SEQ ID NO: 2, amino acids 1 to 218 of SEQ ID NO: 3, amino acids 1 to 208 of SEQ ID NO: 4, amino acids 1 to 215 of SEQ ID NO: 5, amino acids 1 to 207 of SEQ ID NO: 6, amino acids 1 to 201 of SEQ ID NO: 7, amino acids 1 to 201 of SEQ ID NO: 8, amino acids 1 to 203 of SEQ ID NO: 9, amino acids 1 to 208 of SEQ ID NO: 10, amino acids 1 to 207 of SEQ ID NO: 11, amino acids 1 to 208 of SEQ ID NO: 12, amino acids 1 to 207 of SEQ ID NO: 13, amino acids 1 to 207 of SEQ ID NO: 14, amino acids 1 to 207 of SEQ ID NO: 15, amino acids 1 to 208 of SEQ ID NO: 16, amino acids 1 to 208 of SEQ ID NO: 17, amino acids 1 to 206 of SEQ ID NO: 18, amino acids 1 to 207 of SEQ ID NO: 19, amino acids 1 to 216 of SEQ ID NO: 20, amino acids 1 to 218 of SEQ ID NO: 21, amino acids 1 to 204 of SEQ ID NO: 22, amino acids 1 to 203 of SEQ ID NO: 23, amino acids 1 to 208 of SEQ ID NO: 24, amino acids 1 to 210 of SEQ ID NO: 25, amino acids 1 to 207 of SEQ ID NO: 26, amino acids 1 to 207 of SEQ ID NO: 27, amino acids 1 to 208 of SEQ ID NO: 28, amino acids 1 to 217 of SEQ ID NO: 29, amino acids 1 to 208 of SEQ ID NO: 30, amino acids 1 to 201 of SEQ ID NO: 31, amino acids 1 to 202 of SEQ ID NO: 32, amino acids 1 to 207 of SEQ ID NO: 33, amino acids 1 to 202 of SEQ ID NO: 34, amino acids 1 to 201 of SEQ ID NO: 35, amino acids 1 to 202 of SEQ ID NO: 36, amino acids 1 to 206 of SEQ ID NO: 37, amino acids 1 to 202 of SEQ ID NO: 38, amino acids 1 to 202 of SEQ ID NO: 39, amino acids 1 to 202 of SEQ ID NO: 40, amino acids 1 to 202 of SEQ ID NO: 41, amino acids 1 to 206 of SEQ ID NO: 42, amino acids 1 to 207 of SEQ ID NO: 43, amino acids 1 to 208 of SEQ ID NO: 44, amino acids 1 to 215 of SEQ ID NO: 45, amino acids 1 to 217 of SEQ ID NO: 46, amino acids 1 to 214 of SEQ ID NO: 47, amino acids 1 to 208 of SEQ ID NO: 48, amino acids 1 to 203 of SEQ ID NO: 49, amino acids 1 to 216 of SEQ ID NO: 50, amino acids 1 to 207 of SEQ ID NO: 51, amino acids 1 to 208 of SEQ ID NO: 52, amino acids 1 to 207 of SEQ ID NO: 53, amino acids 1 to 208 of SEQ ID NO: 54, amino acids 1 to 207 of SEQ ID NO: 55, amino acids 1 to 207 of SEQ ID NO: 56, amino acids 1 to 208 of SEQ ID NO: 57, amino acids 1 to 207 of SEQ ID NO: 58, amino acids 1 to 207 of SEQ ID NO: 59, amino acids 1 to 207 of SEQ ID NO: 60, amino acids 1 to 204 of SEQ ID NO: 61, amino acids 1 to 216 of SEQ ID NO: 62, amino acids 1 to 245 of SEQ ID NO: 63, amino acids 1 to 249 of SEQ ID NO: 64, amino acids 1 to 248 of SEQ ID NO: 65, amino acids 1 to 245 of SEQ ID NO: 66, amino acids 1 to 249 of SEQ ID NO: 67, amino acids 1 to 245 of SEQ ID NO: 68, amino acids 1 to 247 of SEQ ID NO: 69, amino acids 1 to 250 of SEQ ID NO: 70, amino acids 1 to 240 of SEQ ID NO: 71.

Obtained or obtainable from: The term “obtained or obtainable from” means that the polypeptide may be found in an organism from a specific taxonomic rank. Preferably, the polypeptide is obtained or obtainable from the kingdom Fungi, wherein the term kingdom is the taxonomic rank. More preferably, the polypeptide is obtained or obtainable from the phylum Ascomycota, wherein the term phylum is the taxonomic rank. More preferably, the polypeptide is obtained or obtainable from the subphylum Pezizomycotina, wherein the term subphylum is the taxonomic rank. More preferably, the polypeptide is obtained or obtainable from the class Eurotiomycetes, wherein the term class is the taxonomic rank.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. The skilled person can also compare the sequence to those of the application as filed. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Roughage: The term “roughage” means dry plant material with high levels of fiber, such as fiber, bran, husks from seeds and grains and crop residues (such as stover, copra, straw, chaff, sugar beet waste).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the-nobrief option) is used as the percent identity and is calculated as follows:

(Identical Residues×100)/(Length of Alignment−Total Number of Gaps in Alignment)

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Variant: The term “variant” means a polypeptide having muramidase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In the present invention, a muramidase variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the muramidase activity of the parent muramidase, such as SEQ ID NO: 1 or SEQ ID NO: 63.

Essential Oils: The term “essential oils” means a concentrated liquid containing aroma compounds, obtained by steam distillation from plants. Example of the essential oils include but are not limited to guajacol, turmeric, 3-methylphenol, eugenol, eucalyptol, L-menthol, thymol, capsaicin, tannin, piperine, caravacrol, trimethylamine, 3,4-xylenol, furfuryl alcohol, propylidene, butylidene, phtalides, gingerol, lavender oil, deca-/undeca-dodecalactones, ionones, irone, resorcinol, peppermint oil, alpha-pinene; limonene, anethol, linalool, methyl dihydrojasmonate, carvacrol, propionic, acetic or butyric acid, rosemary oil, clove oil, geraniol, terpineol, citronellol, amyl and/or benzyl salicylate, cinnamaldehyde, vanilline, a plant polyphenol (tannin) and mixtures thereof.

### DETAILED DESCRIPTION OF THE INVENTION

### Composition

It has been surprisingly found that a composition comprising a muramidase (preferably a fungal muramidase) and an essential oil gives an additional performance benefit in animals.

Thus, in a first aspect, the invention relates to a composition comprising one or more polypeptides having muramidase activity and one or more essential oils.

In the present invention, the muramidase may be a GH24 muramidase, preferably a fungal GH24 muramidase, preferably obtained or obtainable from the phylum Ascomycota, more preferably from the class Eurotiomycetes. The muramidase may also be a GH25 muramidase, preferably a fungal GH25 muramidase, preferably obtained or obtainable from the phylum Ascomycota, more preferably from the class Eurotiomycetes.

In the present invention, the essential oil may be thymol, eugenol, meta-cresol, vaniline, salicylate, resorcine, guajacol, gingerol, 3-methylphenol, L-menthol, capsaicin, piperine, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric, curcuma extract and/or mixture thereof.

Preferably, the muramidase is a GH24 muramidase, more preferably a fungal GH24 muramidase, and the essential oil is guajacol, turmeric, 3-methylphenol, eugenol, eucalyptol, L-menthol, thymol, capsaicin, tannin, piperine or mixture thereof.

Preferably, the muramidase is a GH25 muramidase, preferably a fungal GH25 muramidase, and the essential oil is guajacol, turmeric, 3-methylphenol, eugenol, eucalyptol, L-menthol, thymol, capsaicin, tannin, piperine or mixture thereof.

Preferably, the invention relates to a composition comprising one or more polypeptides having muramidase activity and one or more essential oils, wherein the polypeptide having muramidase activity activity is selected from the group consisting of:
(a) a polypeptide having at least 80%, e.g., at least 85%, at least 90%, at least 95%, or 100% sequence identity to SEQ ID NO: 1;
(b) a polypeptide having at least 80%, e.g., at least 85%, at least 90%, at least 95%, or 100% sequence identity to SEQ ID NO: 2;
(c) a polypeptide having at least 80%, e.g., at least 85%, at least 90%, at least 95%, or 100% sequence identity to SEQ ID NO: 3;
(d) a polypeptide having at least 80%, e.g., at least 85%, at least 90%, at least 95%, or 100% sequence identity to SEQ ID NO: 4;
(e) a polypeptide having at least 80%, e.g., at least 85%, at least 90%, at least 95%, or 100% sequence identity to SEQ ID NO: 5;
(f) a polypeptide having at least 80%, e.g., at least 85%, at least 90%, at least 95%, or 100% sequence identity to SEQ ID NO: 6;
(g) a polypeptide having at least 80%, e.g., at least 85%, at least 90%, at least 95%, or 100% sequence identity to SEQ ID NO: 7;
(h) a polypeptide having at least 80%, e.g., at least 85%, at least 90%, at least 95%, or 100% sequence identity to SEQ ID NO: 8;
(i) a polypeptide having at least 80%, e.g., at least 85%, at least 90%, at least 95%, or 100% sequence identity to SEQ ID NO: 9;
(j) a polypeptide having at least 80%, e.g., at least 85%, at least 90%, at least 95%, or 100% sequence identity to SEQ ID NO: 10;
(k) a polypeptide having at least 80%, e.g., at least 85%, at least 90%, at least 95%, or 100% sequence identity to SEQ ID NO: 11;
(l) a polypeptide having at least 80%, e.g., at least 85%, at least 90%, at least 95%, or 100% sequence identity to SEQ ID NO: 12;
(m) a polypeptide having at least 80%, e.g., at least 85%, at least 90%, at least 95%, or 100% sequence identity to SEQ ID NO: 13;
(n) a polypeptide having at least 80%, e.g., at least 85%, at least 90%, at least 95%, or 100% sequence identity to SEQ ID NO: 14;
(o) a polypeptide having at least 80%, e.g., at least 85%, at least 90%, at least 95%, or 100% sequence identity to SEQ ID NO: 15;
(p) a polypeptide having at least 80%, e.g., at least 85%, at least 90%, at least 95%, or 100% sequence identity to SEQ ID NO: 16;
(q) a polypeptide having at least 80%, e.g., at least 85%, at least 90%, at least 95%, or 100% sequence identity to SEQ ID NO: 17;
(r) a polypeptide having at least 80%, e.g., at least 85%, at least 90%, at least 95%, or 100% sequence identity to SEQ ID NO: 18;
(s) a polypeptide having at least 80%, e.g., at least 85%, at least 90%, at least 95%, or 100% sequence identity to SEQ ID NO: 19;
(t) a polypeptide having at least 80%, e.g., at least 85%, at least 90%, at least 95%, or 100% sequence identity to SEQ ID NO: 20;
(u) a polypeptide having at least 80%, e.g., at least 85%, at least 90%, at least 95%, or 100% sequence identity to SEQ ID NO: 21;
(v) a polypeptide having at least 80%, e.g., at least 85%, at least 90%, at least 95%, or 100% sequence identity to SEQ ID NO: 22;
(w) a polypeptide having at least 80%, e.g., at least 85%, at least 90%, at least 95%, or 100% sequence identity to SEQ ID NO: 23;
(x) a polypeptide having at least 80%, e.g., at least 85%, at least 90%, at least 95%, or 100% sequence identity to SEQ ID NO: 24;
v) a polypeptide having at least 80%, e.g., at least 85%, at least 90%, at least 95%, or 100% sequence identity to SEQ ID NO: 25;
(z) a polypeptide having at least 80%, e.g., at least 85%, at least 90%, at least 95%, or 100% sequence identity to SEQ ID NO: 26;
(aa) a polypeptide having at least 80%, e.g., at least 85%, at least 90%, at least 95%, or 100% sequence identity to SEQ ID NO: 27;
(ab) a polypeptide having at least 80%, e.g., at least 85%, at least 90%, at least 95%, or 100% sequence identity to SEQ ID NO: 28;
(ac) a polypeptide having at least 80%, e.g., at least 85%, at least 90%, at least 95%, or 100% sequence identity to SEQ ID NO: 29;
(ad) a polypeptide having at least 80%, e.g., at least 85%, at least 90%, at least 95%, or 100% sequence identity to SEQ ID NO: 30;
(ae) a polypeptide having at least 80%, e.g., at least 85%, at least 90%, at least 95%, or 100% sequence identity to SEQ ID NO: 31;
(af) a polypeptide having at least 80%, e.g., at least 85%, at least 90%, at least 95%, or 100% sequence identity to SEQ ID NO: 32;
(ag) a polypeptide having at least 80%, e.g., at least 85%, at least 90%, at least 95%, or 100% sequence identity to SEQ ID NO: 33;
(ah) a polypeptide having at least 80%, e.g., at least 85%, at least 90%, at least 95%, or 100% sequence identity to SEQ ID NO: 34;
(ai) a polypeptide having at least 80%, e.g., at least 85%, at least 90%, at least 95%, or 100% sequence identity to SEQ ID NO: 35;
(aj) a polypeptide having at least 80%, e.g., at least 85%, at least 90%, at least 95%, or 100% sequence identity to SEQ ID NO: 36;
(ak) a polypeptide having at least 80%, e.g., at least 85%, at least 90%, at least 95%, or 100% sequence identity to SEQ ID NO: 37;
(al) a polypeptide having at least 80%, e.g., at least 85%, at least 90%, at least 95%, or 100% sequence identity to SEQ ID NO: 38;
(am) a polypeptide having at least 80%, e.g., at least 85%, at least 90%, at least 95%, or 100% sequence identity to SEQ ID NO: 39;
(an) a polypeptide having at least 80%, e.g., at least 85%, at least 90%, at least 95%, or 100% sequence identity to SEQ ID NO: 40;
(ao) a polypeptide having at least 80%, e.g., at least 85%, at least 90%, at least 95%, or 100% sequence identity to SEQ ID NO: 41;
(ap) a polypeptide having at least 80%, e.g., at least 85%, at least 90%, at least 95%, or 100% sequence identity to SEQ ID NO: 42;
(aq) a polypeptide having at least 80%, e.g., at least 85%, at least 90%, at least 95%, or 100% sequence identity to SEQ ID NO: 43;
(ar) a polypeptide having at least 80%, e.g., at least 85%, at least 90%, at least 95%, or 100% sequence identity to SEQ ID NO: 44;
(as) a polypeptide having at least 80%, e.g., at least 85%, at least 90%, at least 95%, or 100% sequence identity to SEQ ID NO: 45;
(at) a polypeptide having at least 80%, e.g., at least 85%, at least 90%, at least 95%, or 100% sequence identity to SEQ ID NO: 46;
(au) a polypeptide having at least 80%, e.g., at least 85%, at least 90%, at least 95%, or 100% sequence identity to SEQ ID NO: 47;
(av) a polypeptide having at least 80%, e.g., at least 85%, at least 90%, at least 95%, or 100% sequence identity to SEQ ID NO: 48;
(aw) a polypeptide having at least 80%, e.g., at least 85%, at least 90%, at least 95%, or 100% sequence identity to SEQ ID NO: 49;
(ax) a polypeptide having at least 80%, e.g., at least 85%, at least 90%, at least 95%, or 100% sequence identity to SEQ ID NO: 50;
(ay) a polypeptide having at least 80%, e.g., at least 85%, at least 90%, at least 95%, or 100% sequence identity to SEQ ID NO: 51;
(az) a polypeptide having at least 80%, e.g., at least 85%, at least 90%, at least 95%, or 100% sequence identity to SEQ ID NO: 52;
(ba) a polypeptide having at least 80%, e.g., at least 85%, at least 90%, at least 95%, or 100% sequence identity to SEQ ID NO: 53;
(bb) a polypeptide having at least 80%, e.g., at least 85%, at least 90%, at least 95%, or 100% sequence identity to SEQ ID NO: 54;
(bc) a polypeptide having at least 80%, e.g., at least 85%, at least 90%, at least 95%, or 100% sequence identity to SEQ ID NO: 55;
(bd) a polypeptide having at least 80%, e.g., at least 85%, at least 90%, at least 95%, or 100% sequence identity to SEQ ID NO: 56;
(be) a polypeptide having at least 80%, e.g., at least 85%, at least 90%, at least 95%, or 100% sequence identity to SEQ ID NO: 57;
(bf) a polypeptide having at least 80%, e.g., at least 85%, at least 90%, at least 95%, or 100% sequence identity to SEQ ID NO: 58;
(bg) a polypeptide having at least 80%, e.g., at least 85%, at least 90%, at least 95%, or 100% sequence identity to SEQ ID NO: 59;
(bh) a polypeptide having at least 80%, e.g., at least 85%, at least 90%, at least 95%, or 100% sequence identity to SEQ ID NO: 60;
(bi) a polypeptide having at least 80%, e.g., at least 85%, at least 90%, at least 95%, or 100% sequence identity to SEQ ID NO: 61;
(bj) a polypeptide having at least 80%, e.g., at least 85%, at least 90%, at least 95%, or 100% sequence identity to SEQ ID NO: 62;
(bk) a polypeptide having at least 80%, e.g., at least 85%, at least 90%, at least 95%, or 100% sequence identity to SEQ ID NO: 63;
(bl) a polypeptide having at least 80%, e.g., at least 85%, at least 90%, at least 95%, or 100% sequence identity to SEQ ID NO: 64;
(bm) a polypeptide having at least 80%, e.g., at least 85%, at least 90%, at least 95%, or 100% sequence identity to SEQ ID NO: 65;
(bn) a polypeptide having at least 80%, e.g., at least 85%, at least 90%, at least 95%, or 100% sequence identity to SEQ ID NO: 66;
(bo) a polypeptide having at least 80%, e.g., at least 85%, at least 90%, at least 95%, or 100% sequence identity to SEQ ID NO: 67;
(bp) a polypeptide having at least 80%, e.g., at least 85%, at least 90%, at least 95%, or 100% sequence identity to SEQ ID NO: 68;
(bq) a polypeptide having at least 80%, e.g., at least 85%, at least 90%, at least 95%, or 100% sequence identity to SEQ ID NO: 69;
(br) a polypeptide having at least 80%, e.g., at least 85%, at least 90%, at least 95%, or 100% sequence identity to SEQ ID NO: 70;
(bs) a polypeptide having at least 80%, e.g., at least 85%, at least 90%, at least 95%, or 100% sequence identity to SEQ ID NO: 71;
(bt) a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70 or SEQ ID NO: 71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(bu) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) and a N-terminal and/or C-terminal extension of between 1 and 10 amino acids; and
(bv) a fragment of a polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) having muramidase activity and having at least 90% of the length of the mature polypeptide.

Preferably, the invention relates to a composition comprising one or more polypeptides having muramidase activity and one or more essential oils, wherein the essential oil is selected from the group consisting of thymol, eugenol, piperine or mixture thereof.

In the present invention, the muramidase may comprise or consist of amino acids 1 to 208 of SEQ ID NO: 1, amino acids 1 to 213 of SEQ ID NO: 2, amino acids 1 to 218 of SEQ ID NO: 3, amino acids 1 to 208 of SEQ ID NO: 4, amino acids 1 to 215 of SEQ ID NO: 5, amino acids 1 to 207 of SEQ ID NO: 6, amino acids 1 to 201 of SEQ ID NO: 7, amino acids 1 to 201 of SEQ ID NO: 8, amino acids 1 to 203 of SEQ ID NO: 9, amino acids 1 to 208 of SEQ ID NO: 10, amino acids 1 to 207 of SEQ ID NO: 11, amino acids 1 to 208 of SEQ ID NO: 12, amino acids 1 to 207 of SEQ ID NO: 13, amino acids 1 to 207 of SEQ ID NO: 14, amino acids 1 to 207 of SEQ ID NO: 15, amino acids 1 to 208 of SEQ ID NO: 16, amino acids 1 to 208 of SEQ ID NO: 17, amino acids 1 to 206 of SEQ ID NO: 18, amino acids 1 to 207 of SEQ ID NO: 19, amino acids 1 to 216 of SEQ ID NO: 20, amino acids 1 to 218 of SEQ ID NO: 21, amino acids 1 to 204 of SEQ ID NO: 22, amino acids 1 to 203 of SEQ ID NO: 23, amino acids 1 to 208 of SEQ ID NO: 24, amino acids 1 to 210 of SEQ ID NO: 25, amino acids 1 to 207 of SEQ ID NO: 26, amino acids 1 to 207 of SEQ ID NO: 27, amino acids 1 to 208 of SEQ ID NO: 28, amino acids 1 to 217 of SEQ ID NO: 29, amino acids 1 to 208 of SEQ ID NO: 30, amino acids 1 to 201 of SEQ ID NO: 31, amino acids 1 to 202 of SEQ ID NO: 32, amino acids 1 to 207 of SEQ ID NO: 33, amino acids 1 to 202 of SEQ ID NO: 34, amino acids 1 to 201 of SEQ ID NO: 35, amino acids 1 to 202 of SEQ ID NO: 36, amino acids 1 to 206 of SEQ ID NO: 37, amino acids 1 to 202 of SEQ ID NO: 38, amino acids 1 to 202 of SEQ ID NO: 39, amino acids 1 to 202 of SEQ ID NO: 40, amino acids 1 to 202 of SEQ ID NO: 41, amino acids 1 to 206 of SEQ ID NO: 42, amino acids 1 to 207 of SEQ ID NO: 43, amino acids 1 to 208 of SEQ ID NO: 44, amino acids 1 to 215 of SEQ ID NO: 45, amino acids 1 to 217 of SEQ ID NO: 46, amino acids 1 to 214 of SEQ ID NO: 47, amino acids 1 to 208 of SEQ ID NO: 48, amino acids 1 to 203 of SEQ ID NO: 49, amino acids 1 to 216 of SEQ ID NO: 50, amino acids 1 to 207 of SEQ ID NO: 51, amino acids 1 to 208 of SEQ ID NO: 52, amino acids 1 to 207 of SEQ ID NO: 53, amino acids 1 to 208 of SEQ ID NO: 54, amino acids 1 to 207 of SEQ ID NO: 55, amino acids 1 to 207 of SEQ ID NO: 56, amino acids 1 to 208 of SEQ ID NO: 57, amino acids 1 to 207 of SEQ ID NO: 58, amino acids 1 to 207 of SEQ ID NO: 59, amino acids 1 to 207 of SEQ ID NO: 60, amino acids 1 to 204 of SEQ ID NO: 61, amino acids 1 to 216 of SEQ ID NO: 62, amino acids 1 to 245 of SEQ ID NO: 63, amino acids 1 to 249 of SEQ ID NO: 64, amino acids 1 to 248 of SEQ ID NO: 65, amino acids 1 to 245 of SEQ ID NO: 66, amino acids 1 to 249 of SEQ ID NO: 67, amino acids 1 to 245 of SEQ ID NO: 68, amino acids 1 to 247 of SEQ ID NO: 69, amino acids 1 to 250 of SEQ ID NO: 70 or amino acids 1 to 240 of SEQ ID NO: 71.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. Other examples of conservative substitutions are G to A; A to G, S; V to I, L, A, T, S; I to V, L, M; L to I, M, V; M to L, I, V; P to A, S, N; F to Y, W, H; Y to F, W, H; W to Y, F, H; R to K, E, D; K to R, E, D; H to Q, N, S; D to N, E, K, R, Q; E to Q, D, K, R, N; S to T, A; T to S, V, A; C to S, T, A; N to D, Q, H, S; Q to E, N, H, K, R.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

WO 2013/076253 disclosed that amino acid residues D95 and E97 of SEQ ID NO: 8 of WO 2013/076253 are catalytic residues. PCT/CN2017/075960 discloses the catalytic amino acids of 12 GH25 muramidases. This alignment can be used to determine the position of the catalytic amino acids for the claimed muramidases. In one embodiment, no alteration is made to an amino acid corresponding to E97 and D95 when using SEQ ID NO: 39 for numbering. The catalytic amino acids for the GH24 muramidases can be determined by aligning the sequences with known sequences where the catalytic amino acid(s) have already been determined (see www.uniprot.org).

More preferably, the composition of the present invention comprises the muramidase of SEQ ID NO: 1 and the mixture of thymol, eugenol and piperine.

In the present invention, the polypeptide having muramidase activity may be dosed between 0.001% to 25% w/w of the composition, preferably 0.01% to 25% w/w, more preferably 0.05% to 20% w/w, more preferably 0.2% to 15% w/w, even more preferably 0.5% to 15% w/w or most preferably 1.0% to 10% w/w of the composition.

In the present invention, the essential oil may be dosed between 0.01% to 25% w/w of the composition, preferably 0.1% to 25% w/w, more preferably 0.15% to 20% w/w, more preferably 0.2% to 15% w/w, even more preferably 0.5% to 15% w/w or most preferably 1.0% to 10% w/w of the composition.

In the present invention, the essential oil of the composition may be formulated as a solid formulation; the polypeptide having muramidase activity of the composition may be formulated as a solid formulation; or both the essential oil and the polypeptide having muramidase activity of the composition may be formulated as a solid formulation.

In the present invention, the essential oil of the composition may also be formulated as a liquid formulation; the polypeptide having muramidase activity of the composition may also be formulated as a liquid formulation; or both the essential oil and the polypeptide having muramidase activity of the composition may also be formulated as a liquid formulation.

In the present invention, the liquid formulation may further comprise 20%-80% polyol (i.e. total amount of polyol), preferably 25%-75% polyol, more preferably 30%-70% polyol, more preferably 35%-65% polyol or most preferably 40%-60% polyol. Preferably, the liquid formulation comprises 20%-80% polyol, more preferably 25%-75% polyol, more preferably 30%-70% polyol, more preferably 35%-65% polyol or most preferably 40%-60% polyol wherein the polyol is selected from the group consisting of glycerol, sorbitol, propylene glycol (MPG), ethylene glycol, diethylene glycol, triethylene glycol, 1,2-propylene glycol or 1, 3-propylene glycol, dipropylene glycol, polyethylene glycol (PEG) having an average molecular weight below about 600 and polypropylene glycol (PPG) having an average molecular weight below about 600. More perferably, the liquid formulation comprises 20%-80% polyol (i.e. total amount of polyol), more preferably 25%-75% polyol, more preferably 30%-70% polyol, more preferably 35%-65% polyol or most preferably 40%-60% polyol wherein the polyol is selected from the group consisting of glycerol, sorbitol and propylene glycol (MPG).

In the present invention, the liquid formulation may further comprise preservative, preferably selected from the group consisting of sodium sorbate, potassium sorbate, sodium benzoate and potassion benzoate or any combination thereof. Preferably, the liquid formulation comprises 0.02% to 1.5% w/w preservative, more preferably 0.05% to 1.0% w/w preservative or most preferably 0.1% to 0.5% w/w preservative. More preferably, the liquid formulation comprises 0.001% to 2.0% w/w preservative (i.e. total amount of preservative), preferably 0.02% to 1.5% w/w preservative, more preferably 0.05% to 1.0% w/w preservative or most preferably 0.1% to 0.5% w/w preservative wherein the preservative is selected from the group consisting of sodium sorbate, potassium sorbate, sodium benzoate and potassium benzoate or any combination thereof.

In the present invention, the liquid formulation may comprise one or more formulating agents (such as those described herein), preferably a formulating agent selected from the list consisting of glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the list consisting of 1, 2-propylene glycol, 1,3-propylene glycol, sodium sulfate, dextrin, cellulose, sodium thiosulfate, kaolin and calcium carbonate.

In the present invention, the solid formulation may be for example as a granule, spray dried powder or agglomerate (e.g. as disclosed in WO2000/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

Preferably, the formulating agents of the solid formulation are selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. Preferably, the formulating agent is selected from one or more of the following compounds: sodium sulfate, dextrin, cellulose, sodium thiosulfate, magnesium sulfate and calcium carbonate.

Preferably, the composition of the present invention is an enzyme granule comprising the enzymes of the invention optionally combined with one or more additional enzymes. The granule is composed of a core, and optionally one or more coatings (outer layers) surrounding the core.

Typically, the granule size, measured as equivalent spherical diameter (volume based average particle size), of the granule is 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier. Preparation methods include known granule formulation technologies, e.g.:
a) spray dried products, wherein a liquid enzyme-containing solution is atomized in a spray drying tower to form small droplets which during their way down the drying tower dry to form an enzyme-containing particulate material;
b) layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606;
c) absorbed core particles, wherein rather than coating the enzyme as a layer around the core, the enzyme is absorbed onto and/or into the surface of the core. Such a process is described in WO 97/39116.
d) extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme;
e) prilled products, wherein an enzyme-containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique;
f) mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibres to reinforce the particles to give the so-called T-granulate. Reinforced particles, being more robust, release less enzymatic dust.
g) size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons);
h) fluid bed granulation, which involves suspending particulates in an air stream and spraying a liquid onto the fluidized particles via nozzles. Particles hit by spray droplets get wetted and become tacky. The tacky particles collide with other particles and adhere to them and form a granule;
i) the cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% w/w water.

The core may include additional materials such as fillers, fibre materials (cellulose or synthetic fibres), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

In the present invention, the core may comprise a material selected from the group consisting of salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). Preferably, the core comprises a clay mineral such as kaolinite or kaolin.

The core may also include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt and/or wax and/or flour coating, or other suitable coating materials.

The coating may be applied in an amount of at least 0.1% by weight of the core, e.g., at least 0.5%, 1% or 5%. The amount may be at most 100%, 70%, 50%, 40% or 30% by weight of the core.

The coating is preferably at least 0.1 μm thick, particularly at least 0.5 μm, at least 1 μm or at least 5 μm. In some embodiments the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit is encapsulated or enclosed with few or no uncoated areas. The layer or coating should in particular be homogeneous in thickness.

The coating can further contain other materials as known in the art, e.g., fillers, antisticking agents, pigments, dyes, plasticizers and/or binders, such as titanium dioxide, kaolin, calcium carbonate or talc.

Preferably, the enzyme granules of the invention may comprise a core comprising the enzymes of the invention, one or more salt coatings and one or more wax coatings. Examples of enzyme granules with multiple coatings are shown in WO1993/07263, WO1997/23606 and WO2016/149636.

The salt coating may comprise at least 60% by weight of a salt, e.g., at least 65%, at least 70%, at least 75%, at least 80%, at least 85%, at least 90%, at least 95% or at least 99% by weight. The salt coating may be as described in WO1997/05245, WO1998/54980, WO1998/55599, WO2000/70034, WO2006/034710, WO2008/017661, WO2008/017659, WO2000/020569, WO2001/004279, WO1997/05245, WO2000/01793, WO2003/059086, WO2003/059087, WO2007/031483, WO2007/031485, WO2007/044968, WO2013/192043, WO2014/014647 and WO2015/197719 or polymer coating such as described in WO 2001/00042.

The salt in the coating may have a constant humidity at 20° C. above 60%, particularly above 70%, above 80% or above 85%, or it may be another hydrate form of such a salt (e.g., anhydrate).

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, sorbate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

Specific examples of suitable salts are NaCl (CH20° C.=76%), Na2CO3 (CH20° C.=92%), NaNO3 (CH20° C.=73%), Na2HPO4 (CH20° C.=95%), Na3PO4 (CH25° C.=92%), NH4Cl (CH20° C.=79.5%), (NH4) 2HPO4 (CH20° C.=93,0%), NH4H2PO4 (CH20° C.=93.1%), (NH4) 2SO4 (CH20° C.-81.1%), KCl (CH20° C.=85%), K2HPO4 (CH20° C.=92%), KH2PO4 (CH20° C.-96.5%), KNO3 (CH20° C.=93.5%), Na2SO4 (CH20° C.=93%), K2SO4 (CH20° C.-98%), KHSO4 (CH20° C.=86%), MgSO4 (CH20° C.=90%), ZnSO4 (CH20° C.=90%) and sodium citrate (CH25° C.=86%). Other examples include NaH2PO4, (NH4) H2PO4, CuSO4, Mg(NO3)2, magnesium acetate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, sodium acetate, sodium benzoate, sodium citrate, sodium sulfate, zinc acetate, zinc benzoate, zinc carbonate, zinc chloride, zinc citrate and zinc sorbate.

The salt may be in anhydrous form, or it may be a hydrated salt, i.e. a crystalline salt hydrate with bound water(s) of crystallization, such as described in WO 99/32595. Specific examples include anhydrous sodium sulfate (Na2SO4), anhydrous magnesium sulfate (MgSO₄), magnesium sulfate heptahydrate (MgSO4·7H2O), zinc sulfate heptahydrate (ZnSO4·7H2O), sodium phosphate dibasic heptahydrate (Na2HPO4·7H2O), magnesium nitrate hexahydrate (Mg(NO3)2(6H2O)), sodium citrate dihydrate and magnesium acetate tetrahydrate.

The salt coating may comprise a single salt or a mixture of two or more salts. The salt may be water soluble, in particular having a solubility at least 0.1 g in 100 g of water at 20° C., preferably at least 0.5 g per 100 g water, e.g., at least 1 g per 100 g water, e.g., at least 5 g per 100 g water. The salt may be added from a salt solution where the salt is completely dissolved or from a salt suspension wherein the fine particles are less than 50 μm, such as less than 10 μm or less than 5 μm.

A wax coating may comprise at least 60% by weight of a wax, e.g., at least 65%, at least 70%, at least 75%, at least 80%, at least 85%, at least 90%, at least 95% or at least 99% by weight.

Specific examples of waxes are polyethylene glycols; polypropylenes; Carnauba wax; Candelilla wax; bees wax; hydrogenated plant oil or animal tallow such as polyethylene glycol (PEG), methyl hydroxy-propyl cellulose (MHPC), polyvinyl alcohol (PVA), hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil.

The granulate of the present invention may also be produced as a non-dusting granulate, e.g., as disclosed in U.S. Pat. Nos. 4,106,991 and 4,661,452 and may optionally be coated by methods known in the art. The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

Thus, preferably, the present invention provides a granule, which comprises:
(a) a core comprising an essential oil and muramidase according to the invention, and
(b) a coating consisting of one or more layer(s) surrounding the core.

In the present invention, the coating comprises a salt coating as described herein. Preferably, the coating comprises a wax coating as described herein. More preferably, the coating comprises a salt coating followed by a wax coating as described herein. Even more preferably, the essential oil and the polypeptide having muramidase activity are co-granulated.

In the present invention, the composition may further comprise one or more components selected from the list consisting of one or more carriers. The carrier may be selected from the group consisting of water, glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, wheat flour, wheat bran, corn gluten meal, starch, kaolin and cellulose or any combination thereof.

In present invention, the composition may further comprise one or more additional enzymes; one or more eubiotics; one or more vitamins; one or more minerals, and one or more amino acids, as described below.

### Animal Feed

In the second aspect, the present invention relates to an animal feed comprising an animal feed additive, one or more protein sources and one or more energy sources characterised in that the animal feed further comprises one or more polypeptides having muramidase activity and one or more essential oils as defined above.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

An animal feed composition according to the invention has a crude protein content of 50-800 g/kg. The protein source may be vegetable protein source and/or animal protein.

The vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof. The protein content of the vegetable proteins is at least 10, 20, 30, 40, 50, 60, 70, 80, or 90% (w/w).

Preferably, the vegetable protein source may be material from one or more plants of the family Fabaceae, e.g., soybean, lupine, pea, or bean. The vegetable protein source may also be material from one or more plants of the family Chenopodiaceae, e.g. beet, sugar beet, spinach or quinoa. Other examples of vegetable protein sources are rapeseed, and cabbage. In the present invention, soybean is a preferred vegetable protein source. Other examples of vegetable protein sources are cereals such as barley, wheat, rye, oat, maize (corn), rice, and sorghum.

Besides the vegetable protein as defined above, the animal feed of the invention may also contain animal protein, such as Meat and Bone Meal, Feather meal, and/or Fish Meal, typically in an amount of 0-25%. The animal feed of the invention may also comprise Dried Distillers Grains with Solubles (DDGS), typically in amounts of 0-30%.

Preferably, the protein source is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

Furthermore, or in the alternative (to the crude protein content indicated above), the animal feed composition of the invention may have a content of metabolisable energy of 10-30 MJ/kg. In present invention, the energy source may be selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

Furthermore, or in the alternative (to the crude protein content indicated above), the animal feed composition of the invention may have a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular, the content of metabolisable energy, crude protein, calcium, phosphorus, methionine, methionine plus cysteine, and/or lysine may be within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 D A Beekbergen, The Netherlands. Grafisch bedrijf Ponsen & looijen bv, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

Preferably, the animal feed of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

Animal feed can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) essential oil/muramidase/enzyme preparation may also be added before or during the feed ingredient step. Typically a liquid enzyme preparation comprises the essential oil, the muramidase or both the essential oil and muramidase of the invention optionally with a polyol, such as glycerol, ethylene glycol or propylene glycol, and is added after the pelleting step, such as by spraying the liquid formulation onto the pellets. The essential oil and/or muramidase may also be incorporated in a feed additive or premix.

Alternatively, the essential oil/muramidase can be prepared by freezing a mixture of liquid enzyme solution with a bulking agent such as ground soybean meal, and then lyophilizing the mixture.

In present invention, the animal feed may further comprise one or more additional enzymes; one or more eubiotics; one or more vitamins; one or more minerals, and one or more amino acids, as described below.

The final muramidase concentration in the feed is within the range of 100-1000 mg enzyme protein per kg animal feed, such as 200 to 900 mg, 300 to 800 mg, 400 to 700 mg or 500 to 600 mg enzyme protein per kg animal feed, or any combination of these intervals.

The final essential oil concentration in the feed is within the range of 0.1-20 mg per kg animal feed, such as 0.2 to 15 mg, 0.3 to 10 mg per kg animal feed, or any combination of these intervals.

The animal feed of the present invention may be produced by any known process. For example, the animal feed of the present invention is prepared by a process comprising the steps of:
(a) mixing an animal feed additive with one or more protein sources and one or more energy sources;
(b) optionally steam treating the animal feed of (a) followed by pressing the steam treated mixture to form pellets; and
(c) optionally spraying a liquid formulation onto the animal feed of (a) or (b).

In the present process, the polypeptide having muramidase activity may be added in step (a) and the essential oil may be added in step (c). In one embodiment, the polypeptide having muramidase activity is added in step (c) and the essential oil is added in step (a). In one embodiment, the polypeptide having muramidase activity and the essential oil is added in step (a). In one embodiment, the polypeptide having muramidase activity and the essential oil is added in step (c).

In the present process, the animal feed may be pelleted by steam treating the mixture of (a) to obtain a moisture content below 20% by weight of the mixture, and pressing the steam treated mixture to form pellets. Preferably, the animal feed is pelleted by steam treating the mixture of (a) to obtain a moisture content below 20% by weight of the mixture wherein the steam treatment is between 60° C. and 100° C. when measured at the outlet of the conditioner, and pressing the steam treated mixture to form pellets. In the present process, the total residence time in step b) may be between 1 second and 10 minutes. In the present process, the temperature of the pellets after pelleting of the steam treated mixture may be between 70° C. and 105° C.

### Additional Enzymes

In the present invention, the compositions and/or the animal feed described herein may optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, galactanase, mannanase, dextranase, and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition, the animal feed or the animal feed additive of the present invention may also comprise at least one other enzyme selected from the group comprising of acetylxylan esterase (EC 3.1.1.23), acylglycerol lipase (EC 3.1.1.72), alpha-amylase (EC 3.2.1.1), beta-amylase (EC 3.2.1.2), arabinofuranosidase (EC 3.2.1.55), cellobiohydrolases (EC 3.2.1.91), cellulase (EC 3.2.1.4), feruloyl esterase (EC 3.1.1.73), galactanase (EC 3.2.1.89), alpha-galactosidase (EC 3.2.1.22), beta-galactosidase (EC 3.2.1.23), beta-glucanase (EC 3.2.1.6), beta-glucosidase (EC 3.2.1.21), triacylglycerol lipase (EC 3.1.1.3), lysophospholipase (EC 3.1.1.5), muramidase (EC 3.2.1.17), alpha-mannosidase (EC 3.2.1.24), beta-mannosidase (mannanase) (EC 3.2.1.25), phytase (EC 3.1.3.8, EC 3.1.3.26, EC 3.1.3.72), phospholipase A1 (EC 3.1.1.32), phospholipase A2 (EC 3.1.1.4), phospholipase D (EC 3.1.4.4), protease (EC 3.4), pullulanase (EC 3.2.1.41), pectinesterase (EC 3.1.1.11), xylanase (EC 3.2.1.8, EC 3.2.1.136), beta-xylosidase (EC 3.2.1.37), or any combination thereof.

The composition, the animal feed or the animal feed additive of the invention may also comprise a galactanase (EC 3.2.1.89) and a beta-galactosidase (EC 3.2.1.23).

The composition, the animal feed or the animal feed additive of the present invention may also comprise a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Natuphos™ E (BASF), Finase® and Quantum® Blue (AB Enzymes), OptiPhos® (Huvepharma), AveMix® Phytase (Aveve Biochem), Phyzyme® XP (Verenium/DuPont) and Axtra® PHY (DuPont). Other preferred phytases include those described in e.g. WO 98/28408, WO 00/43503, and WO 03/066847.

The composition, the animal feed or the animal feed additive of the present invention may also comprise a xylanase (EC 3.2.1.8). Examples of commercially available xylanases include Ronozyme® WX (DSM Nutritional Products), Econase® XT and Barley (AB Vista), Xylathin® (Verenium), Hostazym® X (Huvepharma), Axtra® XB (Xylanase/beta-glucanase, DuPont) and Axtra® XAP (Xylanase/amylase/protease, DuPont), AveMix® XG 10 (xylanase/glucanase) and AveMix® 02 CS (xylanase/glucanase/pectinase, Aveve Biochem), and Naturgrain (BASF).

The composition, the animal feed or the animal feed additive of the invention may also comprise a protease (EC 3.4). Examples of commercially available proteases include Ronozyme® ProAct (DSM Nutritional Products), Winzyme Pro Plus® (Suntaq International Limited) and Cibenza® DP100 (Novus International).

The composition, the animal feed or the animal feed additive of the invention may also comprise an alpha-amylase (EC 3.2.1.1). Examples of commercially available alpha-amylases include Ronozyme® A and RONOZYME® RumiStar™ (DSM Nutritional Products).

The composition, the animal feed or the animal feed additive of the invention may also comprise a multicomponent enzyme product, such as FRA® Octazyme (Framelco), Ronozyme® G2, Ronozyme® VP and Ronozyme® MultiGrain (DSM Nutritional Products), Rovabio® Excel or Rovabio® Advance (Adisseo).

### Eubiotics

The composition, the animal feed or the animal feed additive of the invention may additionally comprise eubiotics. Eubiotics are compounds which are designed to give a healthy balance of the micro-flora in the gastrointestinal tract. Eubiotics cover a number of different feed additives, such as probiotics, prebiotics, phytogenics (essential oils) and organic acids which are described in more detail below.

### Probiotics

In the present invention, the composition, the animal feed or the animal feed additive may further comprise one or more additional probiotic. In particular, the animal feed composition may further comprise a bacterium from one or more of the following genera: Lactobacillus, Lactococcus, Streptococcus, Bacillus, Enterococcus, Pediococcus, Leuconostoc, Carnobacterium, Propionibacterium, Bifidobacterium, Clostridium and Megasphaera or any combination thereof.

Preferably, the composition, the animal feed or the animal feed additive further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

More preferably, the composition or the animal feed of the present invention further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, DSM 32315, NRRL B-50136, NRRL B-50605, NRRL B-50606, NRRL B-50622 and PTA-7547.

More preferably, the composition or the animal feed of the present invention further comprises a bacterium from one or more of the following strains of Bacillus pumilus: NRRL B-50016, ATCC 700385, NRRL B-50885 or NRRL B-50886.

More preferably, the composition or the animal feed of the present invention further comprises a bacterium from one or more of the following strains of Bacillus lichenformis: NRRL B 50015, NRRL B-50621 or NRRL B-50623.

More preferably, the composition or the animal feed of the present invention further comprises a bacterium from one or more of the following strains of Bacillus amyloliquefaciens: DSM 29869, DSM 29869, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the composition, the animal feed or the animal feed additive is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. Preferably, the bacterial count of each of the bacterial strains in the composition, the animal feed or the animal feed additive is between 1×10⁸ and 1×10¹⁰ CFU/kg of dry matter.

The bacterial count of each of the bacterial strains in the composition, the animal feed or the animal feed additive is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. Preferably, the bacterial count of each of the bacterial strains in the composition, the animal feed or the animal feed additive is between 1×10⁹ and 1×10¹¹ CFU/animal/day. More preferably, the amount of probiotics is 0.001% to 10% by weight of the composition or the animal feed or animal feed additive.

In the present invention, the one or more bacterial strains may be present in the form of a stable spore.

Examples of commercial products are Cylactin® (DSM Nutritional Products), Alterion (Adisseo), Enviva PRO (DuPont Animal Nutrition), Syncra® (mix enzyme+probiotic, DuPont Animal Nutrition), Ecobiol® and Fecinor® (Norel/Evonik) and GutCare® PY1 (Evonik).

### Prebiotics

Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous bacteria that colonize the large bowel by acting as substrate for them. Normally, prebiotics increase the number or activity of bifidobacteria and lactic acid bacteria in the GI tract.

Yeast derivatives (inactivated whole yeasts or yeast cell walls) can also be considered as prebiotics. They often comprise mannan-oligosaccharids, yeast beta-glucans or protein contents and are normally derived from the cell wall of the yeast, Saccharomyces cerevisiae.

In the present invention, the amount of prebiotics may be 0.001% to 10% by weight of the composition. Examples of yeast products are Yang® and Agrimos (Lallemand Animal Nutrition).

### Organic Acids

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are short chain fatty acids (e.g. formic acid, acetic acid, propionic acid, butyric acid), medium chain fatty acids (e.g. caproic acid, caprylic acid, capric acid, lauric acid), di/tri-carboxylic acids (e.g. fumaric acid), hydroxy acids (e.g. lactic acid), aromatic acids (e.g. benzoic acid), citric acid, sorbic acid, malic acid, and tartaric acid or their salt (typically sodium or potassium salt such as potassium diformate or sodium butyrate).

In the present invention, the amount of organic acid may be 0.001% to 10% by weight of the composition. Examples of commercial products are VevoVitall® (DSM Nutritional Products), Amasil®, Luprisil®, Lupro-Grain®, Lupro-Cid®, Lupro-Mix® (BASF), n-Butyric Acid AF (OXEA) and Adimix Precision (Nutriad).

### Amino Acids

The composition or the animal feed of the invention may further comprise one or more amino acids. Examples of amino acids which are used are lysine, alanine, beta-alanine, threonine, methionine and tryptophan. In the present invention, the amount of amino acid may be 0.001% to 10% by weight of the composition or the animal feed.

### Vitamins and Minerals

In the present invention, the composition or the animal feed may include one or more vitamins, such as one or more fat-soluble vitamins and/or one or more water-soluble vitamins. In addition, the composition or the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

In the present invention, the amount of vitamins may be 0.001% to 10% by weight of the composition or the animal feed. Preferably, the amount of minerals is 0.001% to 10% by weight of the composition or the animal feed.

The nutritional requirements of these components (exemplified with poultry and piglets/pigs) are listed in Table A of WO 01/58275. Nutritional requirement means that these components should be provided in the diet in the concentrations indicated.

In the alternative, the composition or the animal feed of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

Preferably, the composition or the animal feed of the invention comprises at least one of the below vitamins, preferably to provide an in-feed-concentration within the ranges specified in the below Table 1 (for piglet diets, and broiler diets, respectively).

TABLE 1
Typical vitamin recommendations

| Vitamin | Piglet diet | | Broiler diet | |
|---|---|---|---|---|
| Vitamin A | 10,000- | IU/kg feed | 8-12,500 | IU/kg feed |
| | 15,000 | | | |
| Vitamin D3 | 1800- | IU/kg feed | 3000- | IU/kg feed |
| | 2000 | | 5000 | |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

### Other Feed Ingredients

The composition or the animal feed of the invention may further comprise colouring agents, stabilisers, growth improving additives and aroma compounds/flavourings, polyunsaturated fatty acids (PUFAs); reactive oxygen generating species, antioxidants, anti-microbial peptides, anti-fungal polypeptides and mycotoxin management compounds.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Antioxidants can be used to limit the number of reactive oxygen species which can be generated such that the level of reactive oxygen species is in balance with antioxidants.

Mycotoxins, such as deoxynivalenol, aflatoxin, zearalenone and fumonisin can be found in animal feed and can result in negative animal performance or illness. Compounds which can manage the levels of mycotoxin, such as via deactivation of the mycotoxin or via binding of the mycotoxin, can be added to the feed to ameliorate these negative effects. Examples of mycotoxin management compounds are Vitafix®, Vitafix Ultra (Nuscience), Mycofix®, Mycofix® Secure, FUMzyme®, Biomin® BBSH, Biomin® MTV (Biomin), Mold-Nil®, Toxy-Nil® and Unike® Plus (Nutriad).

### Methods of Improving Animal Performance

In the third aspect, the invention further relates to a method of improving body weight gain (BWG), feed conversion ratio (FCR), carotenoid absorption and/or intestinal permeability of a mono-gastric animal comprising administering to the animal the composition or the animal feed comprising one or more polypeptides having muramidase activity and one or more essential oils as defined above.

In the present invention, the improvement is compared to the same feed but excluding the muramidase.

In the present invention, the BWG may be improved by at least 0.5%, such as by at least 1.0%, at least 1.5% or at least 2.0%.

In the present invention, the FCR may be improved by at least 1%, such as by at least 1.25%, at least 1.5% or at least 1.75%.

In the present invention, the carotenoid absorption may be improved by at least 1%, such as by at least 1.5%, at least 2.0%, at least 2.5%, at least 3%, at least 3.5%, at least 4% or at least 5%.

In the present invention, the intestinal permeability may be improved by at least 1%, such as by at least 1.5%, at least 2.0%, at least 2.5%, at least 3%, at least 3.5%, at least 4% or at least 5%. In another embodiment, the EPEF is improved by between 1% and 15%, such as between 1% and 12%, between 1% and 10%, 1.5% and 8%, 2.0% and 7%, or any combination of these intervals.

In the present invention, the essential oil may be dosed at a level of 0.1 to 20 mg per kg animal feed, such as 0.2 to 15 mg, 0.3 to 10 mg per kg animal feed, or any combination of these intervals.

In the present invention, the polypeptide having muramidase activity may be dosed at a level of 100 to 1000 mg enzyme protein per kg animal feed, such as 200 to 900 mg, 300 to 800 mg, 400 to 700 mg or 500 to 600 mg enzyme protein per kg animal feed, or any combination of these intervals.

In the present invention, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals such as cats and dogs, fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

### Use in Improving Animal Performance

In the fifth aspect, the invention further relates to use of a composition or an animal feed in improving body weight gain (BWG), feed conversion ratio (FCR), carotenoid absorption and/or intestinal permeability of a mono-gastric animal, wherein the composition and the animal feed comprise one or more polypeptides having muramidase activity and one or more essential oils as defined above.

In the present invention, the improvement is compared to the same feed but excluding the muramidase.

In the present invention, the BWG may be improved by at least 0.5%, such as by at least 1.0%, at least 1.5% or at least 2.0%.

In the present invention, the FCR may be improved by at least 1%, such as by at least 1.25%, at least 1.5% or at least 1.75%.

In the present invention, the carotenoid absorption may be improved by at least 1%, such as by at least 1.5%, at least 2.0%, at least 2.5%, at least 3%, at least 3.5%, at least 4% or at least 5%.

In the present invention, the intestinal permeability may be improved by at least 1%, such as by at least 1.5%, at least 2.0%, at least 2.5%, at least 3%, at least 3.5%, at least 4% or at least 5%. In another embodiment, the EPEF is improved by between 1% and 15%, such as between 1% and 12%, between 1% and 10%, 1.5% and 8%, 2.0% and 7%, or any combination of these intervals.

In the present invention, the essential oil may be dosed at a level of 0.1 to 20 mg per kg animal feed, such as 0.2 to 15 mg, 0.3 to 10 mg per kg animal feed, or any combination of these intervals.

In the present invention, the polypeptide having muramidase activity may be dosed at a level of 100 to 1000 mg enzyme protein per kg animal feed, such as 200 to 900 mg, 300 to 800 mg, 400 to 700 mg or 500 to 600 mg enzyme protein per kg animal feed, or any combination of these intervals.

In the present invention, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

The present invention will be further illustrated by the following examples.

### EXAMPLES

### Example 1: Determination of Muramidase Activity

The activity of muramidase was determined by measuring the decrease (drop) in absorbance/optical density of a solution of suspended Micrococcus lysodeikticus ATTC No. 4698 (Sigma-Aldrich M3770) measured in a microplate reader (Tecan Infinite M200) at 450 nm.

### Preparation of Micrococcus Lysodeikticus Substrate

Before use the cells were suspended in deionized water to a concentration of 10 mg cells/mL and the absorbance/optical density (OD) at 450 nm was measured. The cell suspension was then adjusted so that the cell concentration in the turbidity assay (180 μL buffer+20 μL sample+20 μL substrate) equaled an OD450=1.0. The adjusted cell suspension was then stored at ambient temperature before use. Suspended cells were used within 3 hours.

### Preparation of Citric Acid-Phosphate Buffer pH 4

61.45 mL 0.1 M citric acid was mixed with 38.55 mL 0.2 M disodium hydrogen phosphate, and the pH was adjusted with hydrochloric acid or sodium hydroxide to pH 4.

### Measurement of Muramidase Antimicrobial Activity in the Turbidity Assay

The muramidase sample to be measured was diluted to a concentration of 50 mg enzyme protein/L in deionized water, and kept on ice until use. In a 96 well microtiter plate (Nunc) 180 L citric acid-phosphate buffer pH 4 and 20 μL of the diluted muramidase sample was added and kept cold (5° C.). To start the activity measurement 20 μL of the substrate (Micrococcus lysodeikticus) was added to each well, and kinetic measurement of absorbance at 450 nm was initiated for 1 hour at 37° C. in a microplate reader. The measured absorbance at 450 nm was monitored for each well and over time a drop in absorbance was seen if the muramidase has muramidase activity.

Following incubation, the muramidase activity against Micrococcus lysodeikticus was determined as Δ absorbance at 450 nm (start value-end value) of each well after 1 hour. Significance was calculated using Dunnett's with control test p level 0.05 in JMP® version 12.1.0 statistical software package from SAS Institute Inc.

### Example 2: In Vivo Broiler Trial

### Experimental Design

### Local:

UFPR—Universidade Federal do Paraná-Paraná-Brazil.
Date start: July 2017
Date finish: February 2018.

### Animal and Housing

A total amount of 1000 day-old-chicks (without antibiotics at hatchery), males, Cobb 500, were placed in floor pen boxes in a completely randomized design. There were 4 treatments, 10 replicates per treatment with 25 birds each.

### Treatments:

$$T 1 - Control$$

$$T 2 - T 1 + Muramidase 25. LSU ( F ) / kg ( 392 mg / kg )$$

$$T 3 - T 2 + {C RINA}^{®} Poutry Plus$$

### Diets:

In this trial were used vegetable mash diets, based on corn and soybean meal. The experimental diets will be formulated in accordance to Table 2 for 3 phases: starter (d1-d21), grower (d21-d35) and finisher (d35-d42). Additionally, in all treatments were added: phytase (RONOZYME® HiPhos GT) 1000 FYT/kg (100 ppm), xylanase (RONOZYME WX 2000 CT) 100 FXU/kg (50 ppm) and 40 mg/kg of CAROPHYLL® yellow 10%.

TABLE 2
Composition and nutrient contents of the basal experimental diets

| | Starter | Grower | Finisher |
|---|---|---|---|
| Ingredients, % | 1-21 days | 22-35 days | 36-42 days |
| CORN | 553.130 | 606.860 | 606.140 |
| SOYBEAN MEAL | 388.000 | 336.000 | 314.000 |
| SALT | 4.800 | 4.300 | 4.100 |
| CHOLINE CHLORIDE | 0.800 | 0.600 | 0.500 |
| SOYBEAN OIL | 24.000 | 28.000 | 45.000 |
| DICALCIUM | 10.000 | 6.800 | 4.500 |
| PHOSPHATE | | | |
| LIMESTONE | 12.000 | 10.800 | 9.900 |
| DL-METHIONINE | 2.970 | 2.670 | 2.480 |
| L-LYSINE | 0.910 | 0.590 | 0.030 |
| L-THREONINE | 0.100 | 0.090 | 0.140 |
| PX ROVIMIX | 1.500 | 1.500 | 1.000 |
| AVES (DSM)* | | | |
| PX ROLIGOMIX | 0.500 | 0.500 | 0.500 |
| AVES (DSM)** | | | |
| HiPhos GT | 0.100 | 0.100 | 0.100 |
| RONOZYME | 0.050 | 0.050 | 0.050 |
| WX 2000 | | | |
| BHT | 0.100 | 0.100 | 0.100 |
| CAROPHYLL ® | 0.040 | 0.040 | 0.000 |
| yellow 10% | | | |
| Celite ® | 0.00 | 0.00 | 10.000 |
| Vehicle | 1.000 | 1.000 | 1.440 |
| | 1,000.000 | 1,000.000 | 1,000.000 |
| Calculated analysis, % | | | |
| AMEn, kcal/kg | 3.000.00 | 3.100.00 | 3.200.00 |
| Crude Protein | 22.00 | 20.00 | 19.00 |
| Calcium | 0.94 | 0.80 | 0.70 |
| Av. P | 0.47 | 0.40 | 0.35 |
| Lys dig | 1.20 | 1.05 | 0.95 |
| Met dig | 0.60 | 0.55 | 0.52 |
| Thr dig | 0.78 | 0.71 | 0.68 |
| Trp dig | 0.26 | 0.23 | 0.22 |

*PX ROVIMIX AVES (DSM): Vit. A 9,000,000 UI/kg; Vit. D3 2,500,000 Ul/kg; Vit. E 20,000 Ul/kg; Vit. K3 2,500 mg/kg; Vit. B1 2,000 mg/kg; Vit. B2 6,000 mg/kg; Pantotenic acid 12 g/kg; Vit. B6 3,000 mg/kg; Vit. B12 15,000 mcg/kg; Nicotinic acid 35 g/kg; Folic acid 1,500 mg/kg; Biotin 100 mg/kg; Selenium 250 mg per kg of premix.
**PX ROLIGOMIX AVES (DSM): Iron 100 g/kg; Cooper 20 g/kg; Manganese 130 g/kg; Cobalt 2,000 mg/kg; Zinc 130 mg/kg; lodine 2,000 mg per kg of premix

### Parameters:

1. Performance: body weight, feed intake and feed conversion ratio: weight gains and feed intakes of each pen of birds were determined and calculated for the whole experiment.
2. Blood carotenoids content: total blood carotenoids content (02 birds per replicate at 35 days of age) were measured by iCheck™ CAROTENE used as a gut integrity biomarker.
3. Mucosal permeability model (FITC-d): technique that evaluates intestinal integrity through the passage of markers high molecular weight (fluorescein isothiocyanate dextran-FITC-d) in Imunova laboratory. At 35 days of age, 2 birds per replicate.

### Statistical Analysis

Data were analyzed by ANOVA at 5% probability and means compare by Tukey test.

### Results and Discussion

### Results

### Performance

Performance data, considering the total experimental period in the different treatments are represented in Table 3.

TABLE 3
Performance of broilers in a period of 1-42 days of age.

| | Body Weight | Feed | Feed Conversion |
|---|---|---|---|
| Treatments | Gain (g) | Intake (g) | Ratio (g:g) |
| Control | 2537 ^{b} | 4334 | 1,694 ^{b} |
| 25,000 LSU (F)/kg of | 2567 ^{b} | 4360 | 1,688 ^{ab} |
| Muramidase | | | |
| 25,000 LSU (F)/kg of | 2584 ^{b} | 4312 | 1.659 ^{a} |
| Muramidase + CRINA ® | | | |
| Poultry Plus (CPP) | | | |
| CV (%) | 4.49 | 3.37 | 1.54 |
| P-value | 0.0057 | 0.0505 | 0.0061 |

Birds that received the combination of muramidase and essential oils (CRINA® Poultry Plus) provides higher body weight gain and lower feed conversion ratio compared to the control group and the group received muramidase alone.

### Total Blood Carotenoids Content:

At 35 days of age blood of birds were collected from the different treatments for carotenoid evaluation (Table 4).

TABLE 4
Total carotenoid content in blood of birds
at 35 days of age who received 4 ppm of Apo-ester
in the diet in a different experimental treatment.

| Treatment | Carotenoids (mg/L) |
|---|---|
| Negative Control | 3.31 ^{b} |
| 25,000 LSU (F)/kg of Bond | 3.53 ^{ab} |
| 25,000 LSU (F)/kg of Bond + | 4.11 ^{a} |
| CRINA ® Poultry Plus (CPP) | |
| CV | 21.32 |
| P-value | 0.0103 |

The birds treated with muramidase and essential oils presented higher levels of total carotenoids in the blood compared to control.

### Mucosal Permeability (Intestinal Integrity):

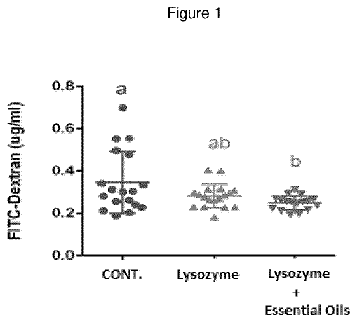

Increased intestinal permeability were measured by levels of serum fluorescein isothiocyanate dextran (FITC-d). There were statistically significant differences between treatment of muramidase+essential oils compare to control (FIG. 1).

### Discussion

The results showed that control group had a worse grow performance compare to treated birds. The addition of muramidase in broiler diets increased weight gain and both, muramidase and the muramidase+essential oils, contributed to better feed conversion ratio.

Better performance in broilers that received an additive in the diet, is closely related to the other results found, like blood carotenoid levels and mucosal permeability, that are directly related to the functionality of TGI and consequently feed efficiency.

Growth performance and feed efficiency are directly related to the intestinal functionality, since the gastrointestinal tract (GIT) is responsible for the nutritional digestion and absorption. Another TGI-related function is the formation of a natural protective barrier, maintained by a balanced dynamic between the mucosal layer, enterocytes, cells of the immune system and by the intestinal microbiota (Sugiharto, 2016).

Intestinal integrity is an indicator of efficiency for the protective barrier formed by TGI, which prevents the paracellular translocation of unwanted compounds such as bacterial toxins and microorganisms from the lumen of the intestine to the lamina propria and later into the blood (Jeurissen et al., 2002). Integrity of the intestinal wall is ensured by a continuous layer of epithelial cells, joined by occlusion junctions, and by a mucosal layer above the epithelium.

Broilers fed with muramidase+essential oils had statistically lower permeabilities than the control, which is an indication of greater integrity of the mucosal barrier (Vicuña et al., 2012). This same treatment also had a greater efficiency in the carotenoid absorption, probably because the treatment prevented more the damage caused by the trial challenges.

### CONCLUSIONS

The inclusion of muramidase and essential oils resulted in better body weight gain, feed conversion compared to control. The improvement in performance may be related to improved gut functionality due to better mucosal integrity index and higher nutrient absorption.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

What is claimed is:

1. A composition comprising one or more polypeptides having muramidase activity and one or more essential oils;
wherein the polypeptide having muramidase activity may be dosed between 0.05% to 20% w/w of the composition, and wherein the essential oil is dosed between 0.01% to 25% w/w of the composition.

2. The composition of claim 1, wherein the polypeptide having muramidase activity is a fungal GH24 muramidase or GH25 muramidase.

3. The composition of claim 1, wherein the essential oil is selected from the group consisting of thymol, eugenol, meta-cresol, vaniline, salicylate, resorcine, guajacol, gingerol, 3-methylphenol, L-menthol, capsaicin, piperine, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric, curcuma extract and/or mixture thereof.

4. The composition of claim 1, wherein the polypeptide having muramidase activity is selected from the group consisting of:
(a) a polypeptide having at least 80%, e.g., at least 85%, at least 90%, at least 95%, or 100% sequence identity to SEQ ID NO: 1;
(b) a polypeptide having at least 80%, e.g., at least 85%, at least 90%, at least 95%, or 100% sequence identity to SEQ ID NO: 2;
(c) a polypeptide having at least 80%, e.g., at least 85%, at least 90%, at least 95%, or 100% sequence identity to SEQ ID NO: 3;
(d) a polypeptide having at least 80%, e.g., at least 85%, at least 90%, at least 95%, or 100% sequence identity to SEQ ID NO: 4;
(e) a polypeptide having at least 80%, e.g., at least 85%, at least 90%, at least 95%, or 100% sequence identity to SEQ ID NO: 5;
(f) a polypeptide having at least 80%, e.g., at least 85%, at least 90%, at least 95%, or 100% sequence identity to SEQ ID NO: 6;
(g) a polypeptide having at least 80%, e.g., at least 85%, at least 90%, at least 95%, or 100% sequence identity to SEQ ID NO: 7;
(h) a polypeptide having at least 80%, e.g., at least 85%, at least 90%, at least 95%, or 100% sequence identity to SEQ ID NO: 8;
(i) a polypeptide having at least 80%, e.g., at least 85%, at least 90%, at least 95%, or 100% sequence identity to SEQ ID NO: 9;
(j) a polypeptide having at least 80%, e.g., at least 85%, at least 90%, at least 95%, or 100% sequence identity to SEQ ID NO: 10;
(k) a polypeptide having at least 80%, e.g., at least 85%, at least 90%, at least 95%, or 100% sequence identity to SEQ ID NO: 11;
(l) a polypeptide having at least 80%, e.g., at least 85%, at least 90%, at least 95%, or 100% sequence identity to SEQ ID NO: 12;
(m) a polypeptide having at least 80%, e.g., at least 85%, at least 90%, at least 95%, or 100% sequence identity to SEQ ID NO: 13;
(n) a polypeptide having at least 80%, e.g., at least 85%, at least 90%, at least 95%, or 100% sequence identity to SEQ ID NO: 14;
(o) a polypeptide having at least 80%, e.g., at least 85%, at least 90%, at least 95%, or 100% sequence identity to SEQ ID NO: 15;
(p) a polypeptide having at least 80%, e.g., at least 85%, at least 90%, at least 95%, or 100% sequence identity to SEQ ID NO: 16;
(q) a polypeptide having at least 80%, e.g., at least 85%, at least 90%, at least 95%, or 100% sequence identity to SEQ ID NO: 17;
(r) a polypeptide having at least 80%, e.g., at least 85%, at least 90%, at least 95%, or 100% sequence identity to SEQ ID NO: 18;
(s) a polypeptide having at least 80%, e.g., at least 85%, at least 90%, at least 95%, or 100% sequence identity to SEQ ID NO: 19;
(t) a polypeptide having at least 80%, e.g., at least 85%, at least 90%, at least 95%, or 100% sequence identity to SEQ ID NO: 20;
(u) a polypeptide having at least 80%, e.g., at least 85%, at least 90%, at least 95%, or 100% sequence identity to SEQ ID NO: 21;
(v) a polypeptide having at least 80%, e.g., at least 85%, at least 90%, at least 95%, or 100% sequence identity to SEQ ID NO: 22;
(w) a polypeptide having at least 80%, e.g., at least 85%, at least 90%, at least 95%, or 100% sequence identity to SEQ ID NO: 23;
(x) a polypeptide having at least 80%, e.g., at least 85%, at least 90%, at least 95%, or 100% sequence identity to SEQ ID NO: 24;
(y) a polypeptide having at least 80%, e.g., at least 85%, at least 90%, at least 95%, or 100% sequence identity to SEQ ID NO: 25;
(z) a polypeptide having at least 80%, e.g., at least 85%, at least 90%, at least 95%, or 100% sequence identity to SEQ ID NO: 26;
(aa) a polypeptide having at least 80%, e.g., at least 85%, at least 90%, at least 95%, or 100% sequence identity to SEQ ID NO: 27;
(ab) a polypeptide having at least 80%, e.g., at least 85%, at least 90%, at least 95%, or 100% sequence identity to SEQ ID NO: 28;
(ac) a polypeptide having at least 80%, e.g., at least 85%, at least 90%, at least 95%, or 100% sequence identity to SEQ ID NO: 29;
(ad) a polypeptide having at least 80%, e.g., at least 85%, at least 90%, at least 95%, or 100% sequence identity to SEQ ID NO: 30;
(ae) a polypeptide having at least 80%, e.g., at least 85%, at least 90%, at least 95%, or 100% sequence identity to SEQ ID NO: 31;
(af) a polypeptide having at least 80%, e.g., at least 85%, at least 90%, at least 95%, or 100% sequence identity to SEQ ID NO: 32;
(ag) a polypeptide having at least 80%, e.g., at least 85%, at least 90%, at least 95%, or 100% sequence identity to SEQ ID NO: 33;
(ah) a polypeptide having at least 80%, e.g., at least 85%, at least 90%, at least 95%, or 100% sequence identity to SEQ ID NO: 34;
(ai) a polypeptide having at least 80%, e.g., at least 85%, at least 90%, at least 95%, or 100% sequence identity to SEQ ID NO: 35;
(aj) a polypeptide having at least 80%, e.g., at least 85%, at least 90%, at least 95%, or 100% sequence identity to SEQ ID NO: 36;
(ak) a polypeptide having at least 80%, e.g., at least 85%, at least 90%, at least 95%, or 100% sequence identity to SEQ ID NO: 37;
(al) a polypeptide having at least 80%, e.g., at least 85%, at least 90%, at least 95%, or 100% sequence identity to SEQ ID NO: 38;
(am) a polypeptide having at least 80%, e.g., at least 85%, at least 90%, at least 95%, or 100% sequence identity to SEQ ID NO: 39;
(an) a polypeptide having at least 80%, e.g., at least 85%, at least 90%, at least 95%, or 100% sequence identity to SEQ ID NO: 40;
(ao) a polypeptide having at least 80%, e.g., at least 85%, at least 90%, at least 95%, or 100% sequence identity to SEQ ID NO: 41;
(ap) a polypeptide having at least 80%, e.g., at least 85%, at least 90%, at least 95%, or 100% sequence identity to SEQ ID NO: 42;
(aq) a polypeptide having at least 80%, e.g., at least 85%, at least 90%, at least 95%, or 100% sequence identity to SEQ ID NO: 43;
(ar) a polypeptide having at least 80%, e.g., at least 85%, at least 90%, at least 95%, or 100% sequence identity to SEQ ID NO: 44;
(as) a polypeptide having at least 80%, e.g., at least 85%, at least 90%, at least 95%, or 100% sequence identity to SEQ ID NO: 45;
(at) a polypeptide having at least 80%, e.g., at least 85%, at least 90%, at least 95%, or 100% sequence identity to SEQ ID NO: 46;
(au) a polypeptide having at least 80%, e.g., at least 85%, at least 90%, at least 95%, or 100% sequence identity to SEQ ID NO: 47;
(av) a polypeptide having at least 80%, e.g., at least 85%, at least 90%, at least 95%, or 100% sequence identity to SEQ ID NO: 48;
(aw) a polypeptide having at least 80%, e.g., at least 85%, at least 90%, at least 95%, or 100% sequence identity to SEQ ID NO: 49;
(ax) a polypeptide having at least 80%, e.g., at least 85%, at least 90%, at least 95%, or 100% sequence identity to SEQ ID NO: 50;
(ay) a polypeptide having at least 80%, e.g., at least 85%, at least 90%, at least 95%, or 100% sequence identity to SEQ ID NO: 51;
(az) a polypeptide having at least 80%, e.g., at least 85%, at least 90%, at least 95%, or 100% sequence identity to SEQ ID NO: 52;
(ba) a polypeptide having at least 80%, e.g., at least 85%, at least 90%, at least 95%, or 100% sequence identity to SEQ ID NO: 53;
(bb) a polypeptide having at least 80%, e.g., at least 85%, at least 90%, at least 95%, or 100% sequence identity to SEQ ID NO: 54;
(bc) a polypeptide having at least 80%, e.g., at least 85%, at least 90%, at least 95%, or 100% sequence identity to SEQ ID NO: 55;
(bd) a polypeptide having at least 80%, e.g., at least 85%, at least 90%, at least 95%, or 100% sequence identity to SEQ ID NO: 56;
(be) a polypeptide having at least 80%, e.g., at least 85%, at least 90%, at least 95%, or 100% sequence identity to SEQ ID NO: 57;
(bf) a polypeptide having at least 80%, e.g., at least 85%, at least 90%, at least 95%, or 100% sequence identity to SEQ ID NO: 58;
(bg) a polypeptide having at least 80%, e.g., at least 85%, at least 90%, at least 95%, or 100% sequence identity to SEQ ID NO: 59;
(bh) a polypeptide having at least 80%, e.g., at least 85%, at least 90%, at least 95%, or 100% sequence identity to SEQ ID NO: 60;
(bi) a polypeptide having at least 80%, e.g., at least 85%, at least 90%, at least 95%, or 100% sequence identity to SEQ ID NO: 61;
(bj) a polypeptide having at least 80%, e.g., at least 85%, at least 90%, at least 95%, or 100% sequence identity to SEQ ID NO: 62;
(bk) a polypeptide having at least 80%, e.g., at least 85%, at least 90%, at least 95%, or 100% sequence identity to SEQ ID NO: 63;
(bl) a polypeptide having at least 80%, e.g., at least 85%, at least 90%, at least 95%, or 100% sequence identity to SEQ ID NO: 64;
(bm) a polypeptide having at least 80%, e.g., at least 85%, at least 90%, at least 95%, or 100% sequence identity to SEQ ID NO: 65;
(bn) a polypeptide having at least 80%, e.g., at least 85%, at least 90%, at least 95%, or 100% sequence identity to SEQ ID NO: 66;
(bo) a polypeptide having at least 80%, e.g., at least 85%, at least 90%, at least 95%, or 100% sequence identity to SEQ ID NO: 67;
(bp) a polypeptide having at least 80%, e.g., at least 85%, at least 90%, at least 95%, or 100% sequence identity to SEQ ID NO: 68;
(bq) a polypeptide having at least 80%, e.g., at least 85%, at least 90%, at least 95%, or 100% sequence identity to SEQ ID NO: 69;
(br) a polypeptide having at least 80%, e.g., at least 85%, at least 90%, at least 95%, or 100% sequence identity to SEQ ID NO: 70;
(bs) a polypeptide having at least 80%, e.g., at least 85%, at least 90%, at least 95%, or 100% sequence identity to SEQ ID NO: 71;
(bt) a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70 or SEQ ID NO: 71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(bu) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) and a N-terminal and/or C-terminal extension of between 1 and 10 amino acids; and
(bv) a fragment of a polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) having muramidase activity and having at least 90% of the length of the mature polypeptide.

5. The composition of claim 1, wherein the essential oil is selected from the group consisting of thymol, eugenol, piperine or mixture thereof.

6. The composition of claim 1, wherein the muramidase is a polypeptide having at least 80% sequence identity to SEQ ID NO: 1; and the mixture of thymol, eugenol and piperine.

7. The composition of claim 1, which further comprises one or more organic acids such as benzoic acid.

8. The composition of claim 1, which further comprises SEQ ID NO: 1, and the mixture of thymol, eugenol and piperine, and benzoic acid.

9. The composition of claim 1, wherein the essential oil is formulated as a solid formulation or liquid formulation; the polypeptide having muramidase activity is formulated as a solid formulation or liquid formulation.

10. The composition of claim 1, which further comprises one or more components selected from the list consisting of one or more carriers; one or more eubiotics, such as probiotics, prebiotics and organic acids; one or more vitamins; one or more minerals; one or more amino acids; and one or more other feed ingredients.

11. An animal feed additive comprising one or more polypeptides having muramidase activity and one or more essential oils;
wherein the polypeptide having muramidase activity may be dosed between 0.05% to 20% w/w of the additive, and wherein the essential oil is dosed between 0.01% to 25% w/w of the additive.

12. An animal feed comprising the composition as defined by any of claims 1 to 10 or the feed additive of claim 11, one or more protein sources and one or more energy sources.

13. The animal feed according to the claim 12, wherein the protein source is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

14. The animal feed of claim 12, wherein the energy source is selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

15. The animal feed of claim 12, which further comprises one or more components selected from the list consisting of:
one or more additional enzymes;
one or more eubiotics, such as probiotics, prebiotics and organic acids;
one or more vitamins;
one or more minerals;
one or more amino acids; and
one or more other feed ingredients.
